(19) United States
(12) Patent Application Publication
(10) Pub. No.: US 2022/0313482 A1
(43) Pub. Date: Oct. 6, 2022

(54) TEMPERATURE MANAGEMENT SYSTEMS AND METHODS FOR EASE AND TIMING OF SETUP AND OPERATION

(71) Applicant: Zoll Circulation, Inc., San Jose, CA (US)

(72) Inventors: Jeremy Thomas Dabrowiak, Santa Clara, CA (US); Sean W. Yip, San Jose, CA (US); Christo Petrov Pamichev, Cupertino, CA (US); Robert J. Smith, Louisville, CO (US); Masoumeh Mafi, Mountain View, CA (US)

(21) Appl. No.: 17/561,512

(22) Filed: Dec. 23, 2021

Related U.S. Application Data
(60) Provisional application No. 63/169,156, filed on Mar. 31, 2021.

(51) Int. Cl. A61F 7/12

(52) U.S. Cl. CPC A61F 7/12; A61F 2007/126; A61F 7/123; A61F 2007/0054

(57) ABSTRACT

A temperature management system for ease and timing of priming, setup and/or operation by a user. The temperature management system is configured to control a temperature of a patient's body using a heat exchange device. The temperature management system includes a console that is coupled to a heat exchange device, such as a catheter or surface pad. The console includes a hardware interface for receiving or coupling to a fluid loop configured to supply the heat exchange device with heating or cooling working fluid (e.g., saline). The interface provides instructions, indicators, and/or hardware elements to assist a user in configuring and priming the fluid loop for operation.

### CLAIM OF PRIORITY

This application claims priority under 35 U.S.C. §119(e) to U.S. Patent Application Ser. No. 63/169,156, filed on Mar. 31, 2021, the entire contents of which are hereby incorporated by reference.

### TECHNICAL FIELD

The present disclosure relates generally to the fields of medicine and engineering and more particularly to improved devices, systems and methods for controlling a patient's body temperature.

### BACKGROUND

In various clinical situations, it is desirable to warm, cool or otherwise control the body temperature of a subject. For example, hypothermia can be induced in humans and some animals for the purpose of protecting various organs and tissues (e.g., heart, brain, kidneys) against the effects of ischemic, anoxic or toxic insult. For example, animal studies and/or clinical trials suggest that mild hypothermia can have neuroprotective and/or cardioprotective effects in animals or humans who suffer from ischemic cardiac events (e.g., myocardial infract, acute coronary syndromes, etc.), postanoxic coma after cardiopulmonary resuscitation, traumatic brain injury, stroke, subarachnoid hemorrhage, fever and neurological injury.

One method for inducing hypothermia is by intravascular or endovascular temperature management wherein a heat exchange catheter is inserted into a blood vessel and a thermal exchange fluid is circulated through a heat exchanger positioned on the portion of the catheter that is inserted in the blood vessel. As the thermal exchange fluid circulates through the catheter's heat exchanger, it exchanges heat with blood flowing past the heat exchanger in the blood vessel. Such technique can be used to cool the subject's flowing blood thereby resulting in a lowering of the subject's core body temperature to some desired target temperature. Endovascular temperature management is also capable of warming the body and/or of controlling body temperature to maintain a monitored body temperature at some selected temperature. If a controlled rate of re-warming or re-cooling from the selected target temperature is desired, that too can be accomplished by carefully controlling the amount of heat added or removed from the body and thereby controlling the temperature change of the patient.

### SUMMARY

This document describes improved temperature management systems for ease and timing of priming, setup and/or operation by a user. The temperature management system is configured to control a temperature of a patient's body using a heat exchange device. The temperature management system may be configured to monitor how a heat exchange device (such as a catheter, pad, etc.) is operating and/or to control the temperature of the patient's body (also called treatment or temperature management treatment or heat exchange treatment). The temperature management system includes a console that is coupled to a heat exchange device, such as a catheter or surface pad. The console includes an interface for receiving or coupling to a fluid loop configured to supply the heat exchange device with heating or cooling working fluid (e.g., saline). The interface may provide instructions, indicators, hardware elements, and/or a graphical interface to assist a user in configuring and priming the fluid loop for operation. The fluid loop includes a plurality of flexible tubes configured to connect a coil, a spike, an air trap cylinder, a pump tube, and a heat exchange device into a fluid loop.

The implementations described herein can provide one or more advantages. The indicators of the interface provide clear association between portions of a disposable fluid loop and their corresponding locations and/or orientations in the console for interfacing the fluid loop with the console and for priming and operating the fluid loop. The sizes, shapes, colors, and/or contents of the indicators of the console correspond to the respective sizes, shapes, colors, and/or contents of indicators of the fluid loop. This assists users in quickly priming the fluid loop and quickly setting up the fluid loop for operation using a proper sequence of actions.

The console interface includes hardware configured to reduce the time needed for a user to set up the fluid loop, allowing the user to perform other tasks while the fluid loop is set up and primed. For example, an air trap holder is included to secure the air trap in a first orientation to trap air bubbles of the fluid loop during priming. This removes a need for the user to physically hold the air trap during priming. The console interface includes a priming actuator configured to automatically prime the fluid loop with a single press. This removes a need for a user to press and hold an actuator until the fluid loop is primed.

The implementations described herein can include one or more of the following embodiments.

In a general aspect, an improved temperature management system for ease and timing of setup by a user comprises a heat exchange fluid loop; a control console comprising: a heat exchange bath configured to receive at least a portion of the heat exchange fluid loop in a heat exchange cavity and couple with a bath cap to close off the heat exchange cavity; a pump comprising: a rotor; a set of rollers that extend from the rotor; an arcuate raceway that extends around the set of rollers, wherein the raceway is configured to receive a pump tube of the heat exchange fluid loop; a pump knob configured to be receivable in a user's hand and allow for ease of grip and rotation of the pump knob by the user, the pump knob configured to orient the rollers to enable loading and unloading of the tube; an air trap coupled to the fluid loop and configured to couple with the control console via an air trap holder in a first orientation during priming of the heat exchange fluid loop and couple with the control console via an air trap receptacle in a second orientation during operation of the temperature management system; a graphical interface comprising a series of instructions for setting up the heat exchange fluid loop, wherein the series of instructions instruct the user on performing at least the following setup steps: installation of the at least a portion of the heat exchange fluid loop in the heat exchange cavity of the heat exchange bath and closing of the heat exchange cavity, placement of the air trap into the air trap holder in the first orientation, and installation of the pump tube by operation of the pump knob.

In some implementations, the control console further comprises a set of indicators each indicative of an order for interacting with the fluid loop to set up the fluid loop for operation, the set of indicators comprising: a first indicator on the bath cap; a second indicator on the air trap holder; a third indicator on a pump input; a fourth indicator on a pump output; a fifth indicator on the priming actuator; and a sixth indicator on the air trap receptacle.

In some implementations, each indicator of the set of indicators corresponds to the series of instructions including depictions of each of the indicators.

In some implementations, one or more indicators of the set of indicators corresponds to a corresponding indicator on the fluid loop, wherein the one or more indicators of the set of indicators is proximate the corresponding indicator on the fluid loop when the fluid loop is coupled to the control console.

In some implementations, at least one indicator includes a particular color that is different from one or more other indicators of the set of indicators, the particular color indicative of a high-priority action for initializing the fluid loop for operation.

In some implementations, a size or shape of at least one indicator is different from one or more other indicators of the set of indicators, the size or shape being indicative of a high-priority action for initializing the fluid loop for operation.

In some implementations, the second indicator on the air trap holder corresponds to a first marking on the air trap to signal a first orientation of the air trap in the air trap holder.

In some implementations, the sixth indicator on the air trap receptacle corresponds to a second marking on the air trap to signal a second orientation of the air trap in the air trap receptacle, the second orientation being different from the first orientation.

In some implementations, the control console further comprises a priming actuator and the series of instructions instruct the user on performing the following additional setup step: pressing and releasing a priming actuator to activate a priming sequence in response to actuation, wherein the priming sequence terminates after a predetermined amount of time.

In some implementations, the fluid loop is coupled to a spike, and the series of instructions instruct the user on performing the following additional setup step: inserting the spike into a fluid bag to fluidly couple the fluid loop to the fluid bag.

In some implementations, the series of instructions instruct the user on performing the following additional setup step: placing the air trap into the air trap receptacle in the second orientation.

In some implementations, the series of instructions instruct the user on performing the setup steps in the following order: a first step of installing the heat exchange fluid loop and closing off the heat exchange bath; a second step of placing the air trap into the air trap holder in the first orientation; a third step of installing the pump tube by operation of the pump knob; a fourth step of inserting the spike into a fluid bag to fluidly couple the fluid loop to the fluid bag; a fifth step of pressing and releasing a priming actuator to activate a priming sequence in response to actuation, wherein the priming sequence terminates after a predetermined amount of time; and a sixth step of placing the air trap into the air trap receptacle in the second orientation.

In some implementations, the air trap holder is keyed to orient the air trap in the first orientation.

In some implementations, the air trap holder includes a resilient gripping element or finger configured to flex to allow the air trap to be inserted into the air trap holder and to secure the air trap in the air trap holder once the air trap is inserted

In some implementations, the system further comprises a pump lid, the pump lid configured to prevent manipulation of the pump rotor during operation of the pump, the pump lid comprising a marking indicating a handle to remove the pump lid from the pump.

In some implementations, the pump further comprising a pump latch, wherein a first portion of the latch is coupled to a pump body and a corresponding second portion of the latch is coupled to the pump lid, wherein the first portion and the second portion connect to hold the lid closed, and wherein less than 5 pounds (lbs.) of force is required to separate the first portion from the second portion to open the lid.

In some implementations, the bath cap is coupled to the heat exchange bath by a retaining device.

In some implementations, the retaining device is a strap that prevents the bath cap from being separated from the heat exchange bath.

In some implementations, the bath cap is physically coupled to the control console when the bath cap is not closing off the heat exchange cavity.

In some implementations, the system includes a priming actuator configured to prime the fluid loop by causing the pump to circulate fluid through the fluid loop.

In some implementations, an indicator is located on the priming actuator.

In some implementations, the priming actuator is configured to activate a priming sequence in response to actuation, and wherein the priming sequence terminates after a predetermined amount of time.

In some implementations, the priming actuator is configured to activate the priming sequence in response to a single press and release of the priming actuator.

In some implementations, the fluid loop comprises a heat exchange device, and wherein the pump is configured to circulate heat exchange fluid of the fluid loop through the heat exchange device.

In some implementations, the fluid loop comprises a catheter, and wherein the pump is configured to circulate heat exchange fluid of the fluid loop through the catheter.

In some implementations, the fluid loop comprises a surface pad, and wherein the pump is configured to circulate heat exchange fluid of the fluid loop through the surface pad.

In some implementations, the fluid loop comprises disposable tubing for an intravascular fluid loop.

In some implementations, the air trap holder is shaped to receive the air trap and comprises a notch to restrain tubing of the fluid loop.

In some implementations, the air trap comprises an evacuation tube configured to remove air from the air trap.

In some implementations, the control console further comprising an optical sensor, wherein the optical sensor is configured to sense a threshold amount of air present in the air trap when the air trap is positioned in an optical pathway of the optical sensor within the air trap receptacle, and wherein an alarm is activated when an amount of air in the air trap exceeds the threshold amount.

In some implementations, the graphical interface comprises a static display of the series of instructions.

In some implementations, the graphical interface comprises a user interface for displaying and interacting with the series of instructions.

In some implementations, the graphical interface comprises a user interface for displaying and interacting with the series of instructions, wherein the instruction comprise icons or animations.

In some implementations, the pump knob extends from an axis of the rotor and rotates around the axis of the rotor, the axis of the rotor being normal to the flat surface, and wherein the flat surface extends over at least one roller of the set of rollers.

In some implementations, the pump knob circumference edge with multiple indentations is configured to assist a grip for torquing the pump knob.

In some implementations, the pump knob extends at least 1-2 in from an axis of the rotor.

In some implementations, the pump knob requires less than 2 in-lb. of torque to rotate the knob.

In some implementations, the pump knob circumference edge with multiple indentations comprises a plurality of finger slots.

In some implementations, the pump knob comprises a diameter ranging from 2-4 inches.

In some implementations, the pump knob comprises a flat top surface, which is receivable in the palm of the user's hand.

In a general aspect, an improved heat exchange fluid loop for a temperature management system for ease and timing of setup by a user comprising: a fluid loop having a tubing assembly, the tubing assembly comprising: a coil, the coil configured to be disposed in a heat exchange bath; a spike comprising an elongate body extending from a base portion, the elongate body comprising a first lumen configured to remove fluid from a fluid bag into the fluid loop and a second lumen configured to return air to the fluid bag from the fluid loop, and the base portion comprising a grip having an angled profile to facilitate ease of insertion of the elongate body into the fluid bag; an air trap configured to trap air present in the fluid loop; a pump tube configured to interface with a fluid pump to circulate fluid through the fluid loop; a plurality of flexible tubes configured to connect the coil, the spike, the air trap, the pump tube, and a heat exchange device; and one or more indicators on the fluid loop configured to match one or more indicators on a control console for ease of installing the fluid loop.

In some implementations, the coil is metallic.

In some implementations, the system includes a sleeve around one or more tubes of the plurality of flexible tubes, wherein the sleeve is configured to reduce or prevent condensation forming on the one or more tubes caused by fluid in the fluid loop.

In some implementations, the one or more indicators comprise: a first indicator proximate the coil, the first indicator corresponding to an indicator on a bath cap of the heat exchange bath; a second indicator on a first end of the air trap; a third indicator on a second end of the air trap; a fourth indicator proximate a first end of the pump tube; a fifth indicator proximate a second end of the pump tube; and a sixth indicator proximate the spike.

In some implementations, the second indicator includes a first color, and wherein the third indicator includes a second color that is different than the first color, the first color corresponding to an air trap holder indicator on the control console, and the second color corresponding to an air trap receptacle indicator on the control console.

In some implementations, the fourth indicator comprises a first color, wherein the fifth indicator includes a second color that is different than the first color, the first color corresponding to a pump input indicator on the control console, and the second color corresponding to a pump output indicator on the control console.

In some implementations, the spike further comprising: a stepped portion, the elongate body extending from the stepped portion, wherein the elongate body has a smaller diameter than the stepped portion, and the stepped portion has a smaller diameter than the base portion.

In some implementations, the elongate body further comprises: a beveled edge configured to pierce into the fluid bag, wherein the first lumen extends through the base portion to the beveled edge, and wherein the second lumen extends through the base portion parallel to the first lumen, the second lumen being shorter than the first lumen, wherein the first lumen and the second lumen comprise a rounded profile edge to prevent fluid leaking from the base portion during insertion of the elongate body into the fluid bag.

In some implementations, the beveled edge is a first beveled edge having a first angle, and wherein the second lumen terminates in a second beveled edge having a second angle that is different from the first angle.

In some implementations, the grip comprising a sequence of ribs, wherein each rib in the sequence is successively larger to cause the angled profile.

In a general aspect, an improved temperature management system for ease and timing of priming comprising: a heat exchange fluid loop comprising a heat exchange device coupled to fluid tubing; a pump comprising one or more rollers and a raceway, the pump configured to receive the fluid tubing of the fluid loop between the one or more rollers and the raceway; an air trap configured to remove air from the fluid loop; a priming actuator configured to generate a signal in response to a press and release of the priming actuator, wherein the signal is generated after a predefined debounce period; and a processor, a memory storing instructions, and associated circuitry communicatively coupled to the priming actuator, wherein the processor is configured to receive the signal from the priming actuator, and in response to receiving the signal, perform: activating the pump to compress the tube between the one or more rollers and the raceway to cause fluid to flow through the fluid loop to remove the air from the fluid loop, wherein the pump is configured to continue to move fluid through the fluid loop for a predefined period of time after the priming actuator is released.

In some implementations, the priming actuator is configured to generate a signal in response to a single press and release of the priming actuator.

In some implementations, the period of time comprises 1 to 3 minutes.

In some implementations, the debounce period comprises 100-200 milliseconds (ms).

In some implementations, the priming actuator is a button.

In some implementations, the button is illuminated upon actuation and remains illuminated until priming is complete.

In some implementations, the pump is configured to move fluid through the fluid loop until all air in the fluid loop is moved into the air trap.

In some implementations, the operations further comprise: detecting a second signal representing a second actuation of the priming actuator while the pump is moving fluid through the fluid loop; and causing the pump to stop moving fluid through the fluid loop in response to detecting the second signal.

In some implementations, the system includes a bubble sensor configured to detect the air in the fluid loop. The operations further comprise: causing the pump to move fluid through the fluid loop until no air is detected by the bubble sensor for a period of time.

In some implementations, the operations are performed by the processor using discrete logic devices.

In some implementations, the operations are performed by a microprocessor.

In some implementations, the heat exchange device comprises a catheter.

In some implementations, the heat exchange device comprises a surface pad.

In some implementations, the air trap holder in combination with the priming actuator allows the user to perform one or more of the following: step back and observe a priming process, ensuring that bubbles are exiting the fluid loop properly, that there is no leaking of the fluid loop, and leave the system unattended to perform other duties during priming.

In some implementations, the fluid loop comprises a heat exchange device, and wherein the fluid pump is configured to circulate heat exchange fluid of the fluid loop through the heat exchange device.

In some implementations, the fluid loop comprises a catheter, and wherein the fluid pump is configured to circulate heat exchange fluid of the fluid loop through the catheter.

In some implementations, the fluid loop comprises a surface pad, and wherein the fluid pump is configured to circulate heat exchange fluid of the fluid loop through the surface pad.

In some implementations, the air trap holder is an air trap stand.

In some implementations, the pump knob has a circumference edge with multiple indentations and the circumference edge allows the user ease of grip and rotation of the pump knob.

The details of one or more embodiments are set forth in the accompanying drawings and the description below. Other features and advantages will be apparent from the description and drawings, and from the claims.

### BRIEF DESCRIPTION OF THE DRAWINGS

FIG. 1 shows an embodiment of a temperature management system for use with a patient.

FIG. 2 shows a block diagram representing portions of a hardware interface of the temperature management system of FIG. 1.

FIG. 3A shows a perspective view of an interface between the console and a heat exchange fluid loop of the temperature management system of FIG. 1.

FIG. 3B shows a perspective view of the interface of FIG. 3A with the air trap in the air trap holder.

FIG. 3C shows a perspective view of the interface of FIG. 3A showing the bath cavity.

FIG. 3D shows a schematic of the cap of the heat exchange bath.

FIG. 3E shows a perspective view of the interface of FIG. 3A including a user using the pump knob.

FIG. 3F shows a view of the priming actuator of interface 3A.

FIG. 3G shows a perspective view of the interface of FIG. 3A including an alternative air trap holder.

FIG. 4A shows an illustration of a graphical interface with example instructions that correspond to the interface shown in FIG. 3A.

FIG. 4B shows an illustration of example user interface instructions that correspond to the interface shown in FIG. 3A.

FIG. 5 shows an example of a removable heat exchange fluid loop of the temperature management system of FIGS. 2-3.

FIG. 6A shows an example of a spike.

FIG. 6B shows an example of spike lumens of the spike of FIG. 6A.

FIG. 7A shows an example of a pump.

FIG. 7B shows an example of a pump knob.

FIG. 8A shows a flow diagram including an example process for interfacing the fluid loop with the console interface.

FIG. 8B shows a flow diagram including an example process for priming a fluid loop for temperature management treatment.

FIG. 9 is a diagram of an example computing system for executing operations of the temperature management system of FIG. 1.

### DETAILED DESCRIPTION

When a patient in critical condition, e.g., a patient who has experienced cardiac arrest, needs temperature management treatment to control their body temperature, it is necessary to administer such treatment as quickly and efficiently as possible to optimize the effectiveness of the treatment and the patient's chances for recovery. The improved temperature management systems and heat exchange fluid loops described herein include several features that facilitate easy and rapid setup by a caregiver, allowing the caregiver to provide the patient with treatment as quickly and efficiently as possible.

For example, various indicators and graphical interfaces with instructions are described herein that provide step-by-step guidance for quickly installing a heat exchange fluid loop and setting up a system for priming and operation. The indicators and easy to follow instructions and pictorial representations make setup simple and help reduce setup time. Various components are also described herein that facilitate fast and easy connection of the fluid loop to a console of the system and priming of the fluid loop in a manner that free's up the caregiver, so that the caregiver can focus on the patient, and/or handle other treatment preparations.

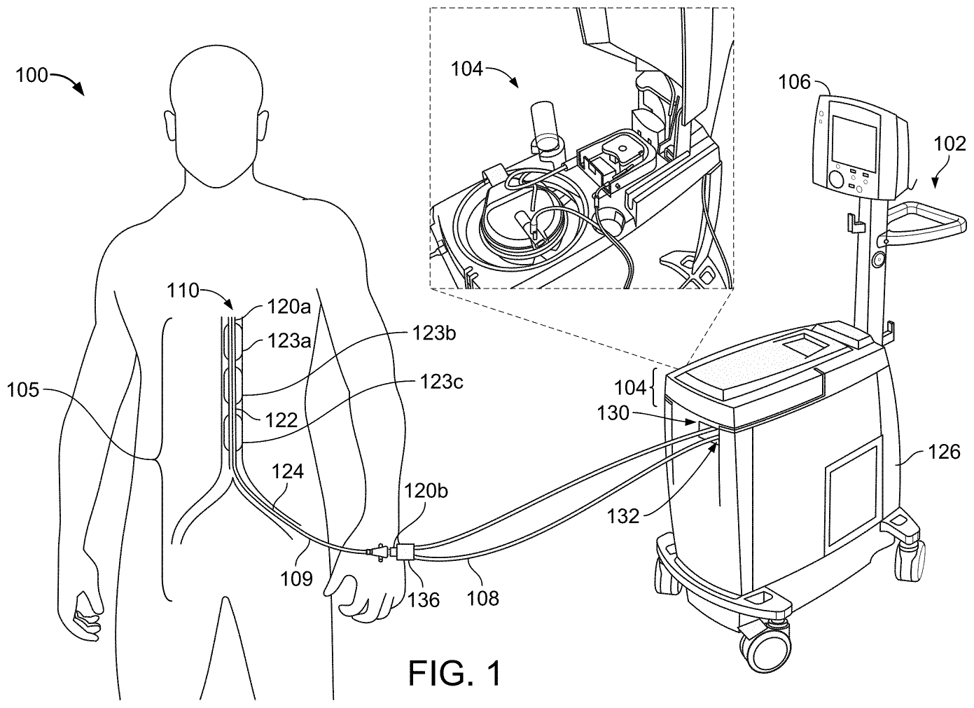

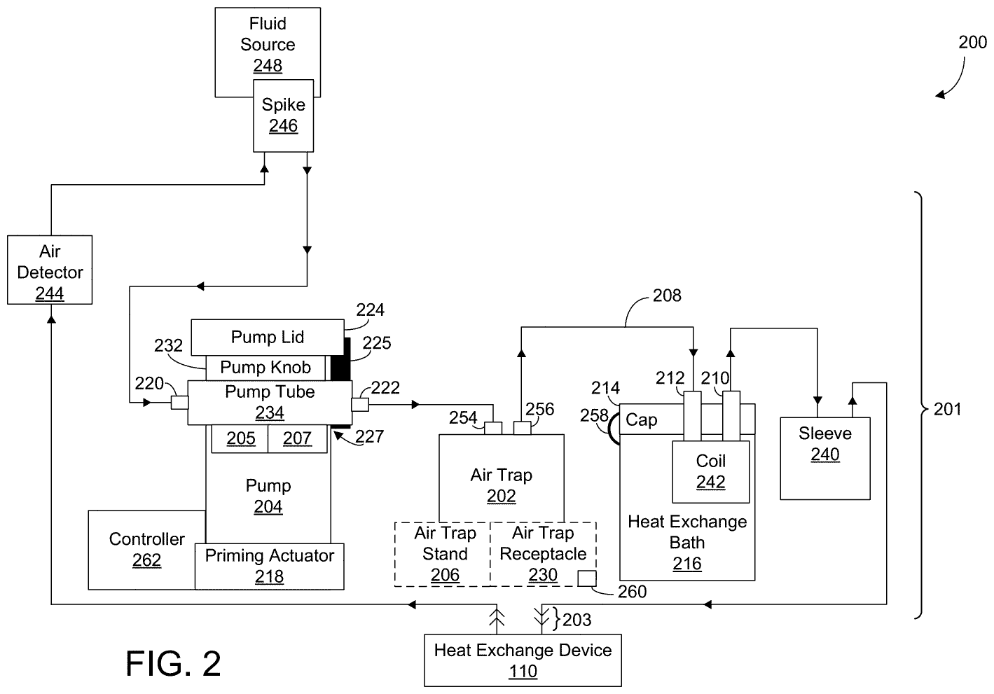

FIG. 1 shows a temperature management system 100. The temperature management system 100 is configured to control a temperature of a patient's body using a heat exchange device 110. The temperature management system 100 is configured to heat or cool the patient (or both) to manage the temperature of the patient. Managing the temperature of the patient may be referred to as heat exchange treatment of the patient, or heating/warming or cooling treatment of the patient. The temperature management system 100 includes a heat exchange device 110, e.g., an intravascular heat exchange catheter configured to be inserted into a vasculature 105 of a patient, or a heat exchanger applied to the surface of a patient, such as a heat exchange pad. The temperature management system 100 can include other hardware configured for heating or cooling the patient, such as heat an exchange fluid loop, heating or cooling plates, heating or cooling cassettes, heat exchange baths, and so forth as subsequently described for heating or cooling the patient (or both). Some of said examples are subsequently described in relation to FIGS. 2-7B.

The temperature management system 100 includes an extracorporeal control console 102 with a hardware interface 104, subsequently described. The interface 104 allows the heat exchange device 110 to be coupled to the control console 102 by interfacing with a heat exchange fluid loop that includes the heat exchange device and a tubing assembly as subsequently described. The control console 102 includes a controller and/or processor for controlling the heat exchange device 110. The control console includes a user interface 106 for allowing a user to input data or control signals to the temperature management system 100 and to present information, such as treatment data, indicative of treatment of the patient using the temperature management system 100. The user interface 106 may present or display various types of information associated with temperature management treatment of the patient and use of the temperature management system, including, e.g., patient data, operational data, priming instructions, and/or setup instructions.

The temperature management system 100 is configured to measure operational data representing operation of one or more aspects of the temperature management system 100. The temperature management system 100 is configured to measure patient data representing one or more physiological parameters of the patient, e.g., patient temperature, during treatment of the patient. The operational data and the patient data that are measured during treatment of the patient may be referred to as treatment data. The temperature management system may be configured to control the temperature of the patient's body based on the operational data (e.g., pump speed, coolant temperature, heat exchange bath temperature, working fluid temperature and power), and/or patient data, (e.g., patient temperature feedback received from temperature sensors located in or on the patient). The temperature management system 100 is configured to display, by a user interface 106, an operational status of the temperature management system 100 and a physiological status of the patient during treatment. The operational status can include whether the temperature management system 100 is working at a maximum cooling or heating power (e.g., effort) or a percentage of the maximum heating or cooling power. The physiological status can include the temperature of the patient.

The user interface 106 is configured to display operational data and patient data on the user interface in a configuration that allows a user to determine a stage of a temperature management treatment (e.g., a treatment cycle or treatment process) being performed on the patient. The user interface 106 shows a current operational treatment period (also called a stage). Each treatment period of the temperature management treatment is associated with a target patient temperature and a rate of cooling or heating the patient to control the patient temperature to the target temperature.

A cooling or heating power exerted by the temperature management system 100 to cool or heat the patient is displayed on the user interface 106. The cooling or heating power (also called “effort” or simply “power”) represents how hard the temperature management system 100 is working to heat or cool the patient. The actual value of the power represents a percentage of a maximum possible cooling or heating rate or capacity or capability of the system to cool or heat the patient based on a relationship between a heat exchange bath or coolant temperature of the temperature management system 100 and the patient's current temperature.

The extracorporeal control console 102 includes hardware for managing the patient temperature. The hardware includes components that make up an interface 104 of the extracorporeal control console 102, which are configured to interface with components of a fluid loop that, and the fluid loop is configurable for connecting to the heat exchange device 110. The fluid loop includes a tubing assembly 108 which facilitates connection of the heat exchange device 110 with the control console 102. One or more temperature sensors 120a, 120b may be located on or in the heat exchange device 110 and/or may be located on a separate device or probe positioned elsewhere in the body, e.g., in the esophagus or rectum of the patient. In some implementations, the heat exchange device 110, fluid loop, and/or the temperature sensors 120a-b may be disposable items intended for a single use. The control console 102 may be a non-disposable system intended for multiple uses and to interface with the single-use device(s).

In the implementation shown, an intravascular heat exchange catheter (which is device 110) comprises an elongate catheter body 122 and one or more heat exchangers 123a-c positioned on a distal portion of the catheter body 122, through which a thermal exchange fluid circulates. The heat exchangers, in the example of FIG. 1, include inflatable cylindrical balloons. In an alternative implementation, the heat exchanger may include a serpentine or helical balloon or tubing (not shown). Inflow and outflow lumens (not shown) are present within the catheter body 122 to facilitate circulation of the thermal exchange fluid (e.g., sterile 0.9% sodium chloride solution or other suitable thermal exchange fluid) through the elongate catheter body 122 and through the heat exchange balloons. Optionally, the catheter body 122 may also include one or more working or through lumens 124 which extend through the catheter body 122 and terminate distally at one or more openings in the distal end of the catheter body. The working lumens can each serve as a guidewire lumen to facilitate insertion and positioning of the catheter and/or may be used after insertion of the catheter for delivery of fluids, medicaments or other devices. For example, as shown in FIG. 1, in some embodiments, the temperature sensors 120a-b may be inserted through the working lumen of the catheter and advanced out of the distal end opening to a location beyond the distal end of the catheter body 122. Alternatively, in other embodiments, the temperature sensors 120a-b may be positioned at various other locations, using a separate device, catheter or probe, on or in the subject's body to sense the desired body temperature(s). Various heat exchange catheters may be used in the embodiments described herein.

Non-limiting examples of various heat exchange devices, heat exchange catheters and/or heat exchange pads or body surface heat exchangers that may be used are described in U.S. Pat. No. 9,492,633, titled Heat exchange catheter and their methods of manufacture and use and issued on Nov. 15, 2016, and U.S. Application Pub. No. 2013/0090708, titled Endovascular Cooling Catheter System Which Employs Phase-Changing Heat Exchange Media and filed on Sep. 28, 2012, U.S. Pat. No. 9,662,243, titled Heat Exchange Catheters with Bi-Directional Fluid Flow and Their Methods of Manufacture and Use and issued on May 30, 2017, U.S. Pat. No. 100,045,881, titled Patient Temperature Control Catheter with Helical Heat Exchange Paths and issued on Aug. 14, 2018, U.S. Pat. No. 9,314,370 titled Self-Centering Patient Temperature Control Catheter and issued on Apr. 19, 2016, U.S. Pat. No. 9,241,827 titled Intravascular Heat Exchange Catheter with Multiple Spaced Apart Discrete Coolant Loops and issued on Jan. 26, 2016, U.S. Pat. No. 9,717,625 titled Intravascular Heat Exchange Catheter with Non-Round Coiled Coolant Path and issued on Aug. 1, 2017, U.S. Pat. No. 9,433,526 titled Intravascular Heat Exchange Catheter With Rib Cage-Like Coolant Path and issued on Sep. 6, 2016, 2018/0185193, titled High Efficiency Heat Exchange Catheters For Control Of Patient Body Temperature and filed on Dec. 30, 2016, U.S. Pat. App. 2018/018519, filed on Dec. 30, 2016, titled Fluid-Circulating Catheters Usable for Endovascular Heat Exchange, and 2018/0207024, titled Managing Patient Body Temperature Using Endovascular Heat Exchange in Combination with Body Surface Heat Exchange and filed on Jan. 23, 2017, the entire disclosure of each such patent and application being expressly incorporated herein by reference. Other examples of catheters that may be used herein include those commercially available from ZOLL Circulation, Inc., San Jose, Calif., such as the Cool Line® Catheter, Icy® Catheter, Quattro® Catheter, and Solex 7® Catheter.

The extracorporeal control console 102 generally comprises a main housing and a console head having a user interface 106. The main housing 126 contains various apparatuses and circuitry for warming/cooling thermal exchange fluid, e.g., coolant, refrigerant, saline, to controlled temperature(s) and for pumping such warmed or cooled thermal exchange fluid through the heat exchange device 110 to effectively modify and/or control the subject's body temperature. The console head includes a display device or user interface 106, such as a touch screen system, whereby certain information may be input by, and certain information may be displayed to, users of the system 100. On the housing 126, there may be provided connection ports 130, 132 for connection of additional or alternative types of temperature sensors and/or other apparatuses. A connector 136 can connect the tubing 109 of the tubing assembly 108 from the console 102 to the inflow and outflow tubes of the heat exchange device 110.

FIG. 2 is a block diagram showing hardware 200 of a control console (e.g., portions of hardware interface 104 of control console 102 of FIG. 1) and a fluid loop 201 configured to interface with the control console 102. The block diagram shows components of the fluid loop 201 interfaced with components of the control console 102. The components of the control console 102 that interface with the fluid loop include a heat exchange bath 216 (e.g., a coldwell), a pump 204 (e.g., a peristaltic pump), an air trap holder 206, an air trap receptacle, a priming button 218 and/or the controller 262. In FIG. 2, the lines with arrows (marked 208) represent tubing configured to accommodate fluid flow.

One or more of the console interface components are configured to engage or couple with one or more components of a heat exchange fluid loop 201 as described in further detail below. The heat exchange fluid loop 201 (also fluid loop 201) includes hardware components that form a path for heat exchange fluid to flow during temperature management treatment by the heat exchange system 100. The heat exchange fluid loop 201 is removable from the console interface. In some implementations, the heat exchange fluid loop 201 is disposable such that it is configured for a single use. In some implementations, the fluid loop 201 components may include a spike 246, a coil 242, an air trap 202, a pump tube 234, a heat exchange device 110, and/or a bubble detector 244, and tubing 208 configured to connect each of these components for fluid flow among each of the components. The tubing 208 may be covered by a sleeve 240 (e.g., a polyurethane sleeve or similar material) configured to assist a user in configuring or arranging the fluid loop, as subsequently described.

The hardware components 200 for temperature management treatment of the patient are configured to circulate heat exchange fluid (not shown) through the patient. A fluid source 248 provides the heat exchange fluid for the system. For example, the fluid source 248 can include a saline bag configured to provide a saline working fluid to the fluid loop 201.

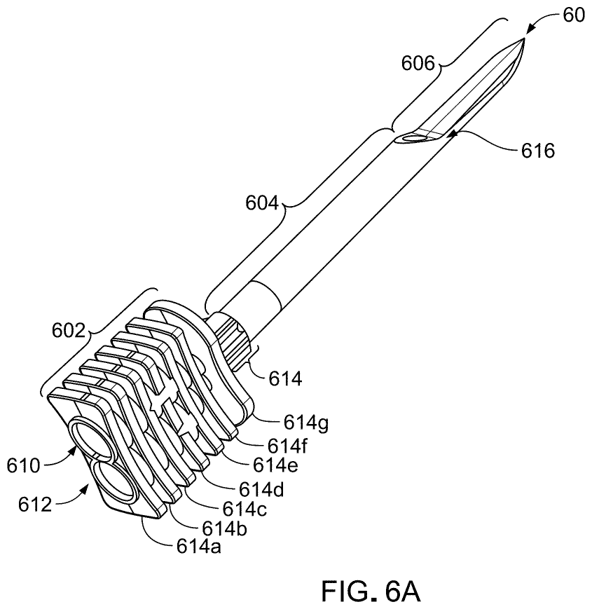

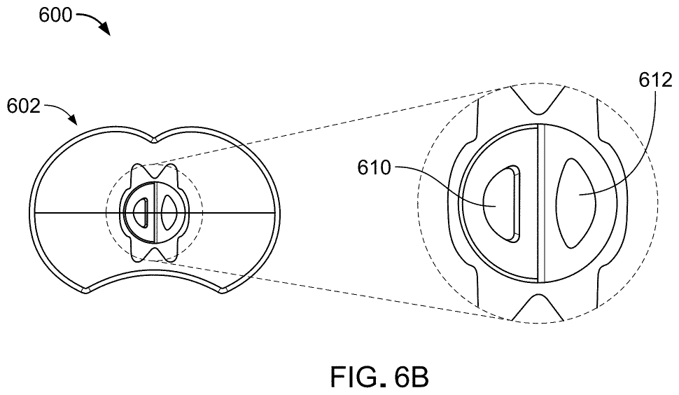

A bag spike 246 (also called a saline spike or simply spike 248) is provided to tap the fluid source and extract fluid from the fluid source. The spike 248 is described in greater detail with respect to FIG. 6A. The spike is configured to puncture a saline bag and drain the saline from the saline bag to fill the fluid loop 201 with working fluid. The fluid source 248 may be placed above the rest of the working fluid loop so that fluid exits the fluid source to prime the fluid loop. The spike 246 includes an elongate body extending from a base portion. The elongate body includes a first inflow lumen configured to remove fluid e.g., liquid, from a fluid bag into the fluid loop and a second outflow lumen configured to return liquid and/or air to the fluid bag from the fluid loop. The base portion includes a grip having an angled profile to facilitate ease of insertion of the elongate body into the fluid bag. The spike 246 is further described in relation to FIG. 6A.

A pump 204 is configured to pump the working fluid through the fluid loop 201. The pump can be a peristaltic pump that engages a pump tube 234 of the fluid loop 201. The pump 204 compresses the pump tube 234 to pump the working fluid through the fluid loop 201.

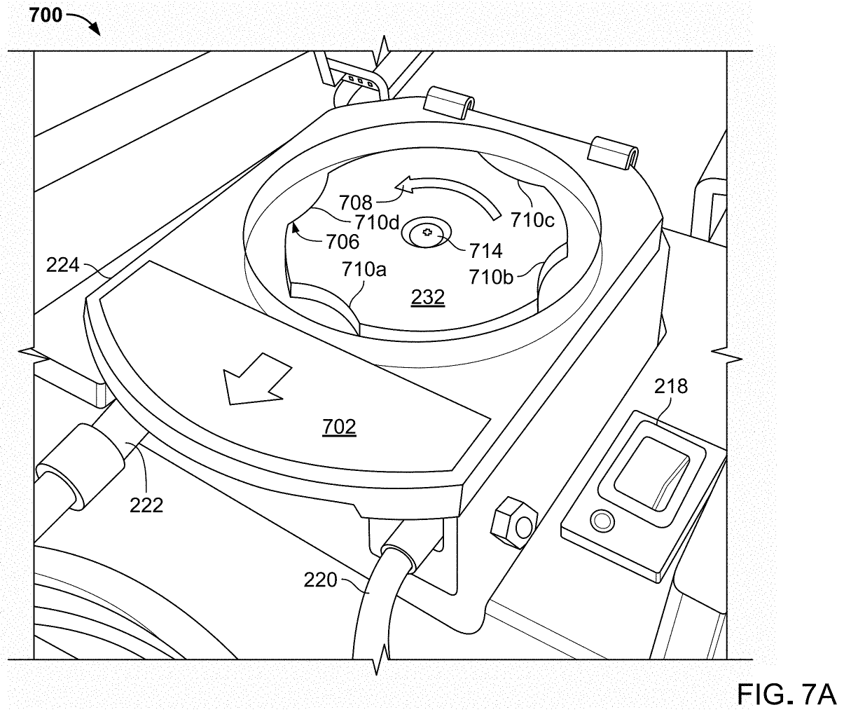

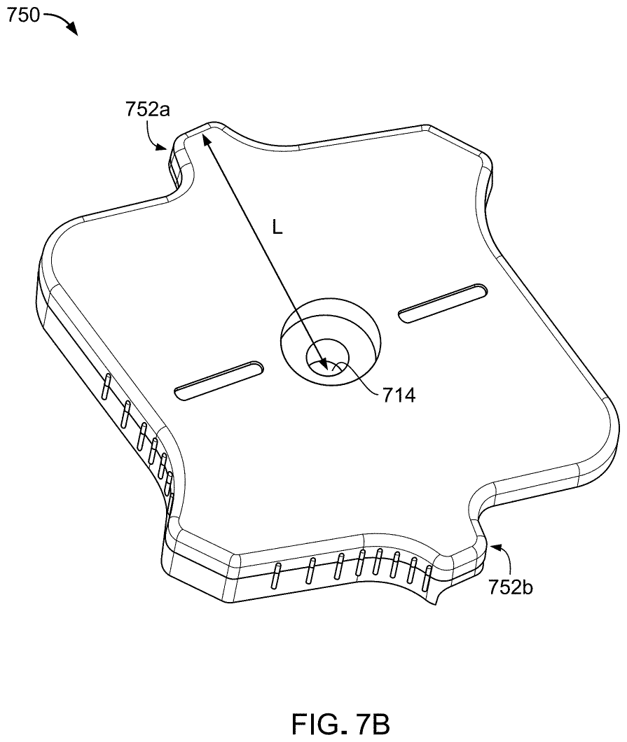

The pump 204 can be accessed by opening a pump lid 224 (also called pump cover) on the console. The pump lid 224 can be lifted and a pump knob (not shown) can be turned to advance or load the pump tube 234 onto the pump 204 and/or help unload or remove the pump tube 234 from the pump 204. The pump lid 224 is configured to prevent manipulation of or inadvertent contact or disruption of the pump rotor during operation of the pump. As seen in FIG. 7A, the pump lid includes an indicator indicating a handle to remove the pump lid 224 from the pump 204 or to rotate the pump lid 224 into an open position to access the pump. The pump 204 includes a pump latch 225. A first portion of the latch 225 is coupled to a pump body 227 of the pump 204. A corresponding second portion of the latch 225 is coupled to the pump lid 224. The first portion and the second portion connect to hold the lid closed. Generally, less than 5 lbs. of force or 2 to 4 lbs of force is required to separate the first portion from the second portion to open the lid.

Continuing with FIG. 2, the pump tube 234 is connected to the tubing 208 by inlet port 220 and outlet port 222. The pump tube 234 can be a different diameter and made of a different material than the tubing 209. The pump tube 234 is configured for compression and expansion in response to contact by rollers 205 of the pump 204. The pump tube 234 is attached to the tubing 208 and is aligned in an arcuate raceway of the pump 204 when the fluid loop 201 is installed on the console. The pump 204 drives fluid flow through the tubing 208, through the air trap cylinder 204, through the coil 242, and to the heat exchange device 110. The pump 204 is controlled by a controller 262 that is configured to control a speed of the pump and thus a rate of the fluid flow through the fluid loop 201. The controller 262 can control a heating or cooling rate of the patient by controlling the temperature of the working fluid and/or a rate of the working fluid flow through the fluid loop 201.

To install the fluid loop 201, the pump lid 224 is opened. The pump tube 234 is fed into the pump 204 between the rollers 205 and the raceway 207 of the pump. The rollers 205 of the pump 204 are advanced while placing the pump tube 234 into the raceway 207 of the pump. A pump knob 232 allows a user to advance the rollers 205 of the pump 204 manually to facilitate positioning of the pump tube 234 into the raceway 207. The pump knob 232 is sized to allow the user to advance the rollers 205 while applying minimal torque to the pump knob. The pump 204 is further described in relation to FIGS. 7A-7B.

The heat exchange fluid loop 201 includes an air trap 202 which is configured for trapping air in the fluid loop 201 during operation of the heat exchange system 100. The air trap 202 includes a chamber for storing fluid and trapping air bubbles from the fluid loop. The air trap 202 is filled with working fluid during priming of the fluid loop. The air trap 204 is held in an inverted position during priming to trap air bubbles. In some implementations, the air trap 202 includes a cylinder. The air trap 202 has an input 254 and an output 256 at an end of the air trap. A first end of tubing 208 (input tubing) enters the air trap through the input 254 and a second end of tubing (output tubing) enters the air trap through output 256, such that both the input and output tubing are in fluid communication with the air trap 202. The end of the air trap having the input 254 and output 256 is at a lowest point on the air trap 202, below the air trap 202, when the air trap is inverted during priming, and at a highest point, above the air trap 202, during temperature management treatment after priming is completed.

The air trap 202 may be connected to an evacuation tube 296. The evacuation tube 296 is tubing configured to remove air from the air trap. The evacuation tube 296 can extend from the air trap 202.

An air trap holder 206 is configured to secure the air trap 202 in the inverted position during priming. In certain implementations, the air trap holder may be a stand, clip, bracket or other structure configured to hold or secure the air trap 202. The air trap holder 206 is shaped to hold the air trap 202 in place during priming. For example, the air trap holder 206 may be keyed to hold the air trap 202 in the inverted orientation during priming. In some implementations, the air trap holder 206 includes a notch or space for routing or restraining the input and output tubing 208 for the air trap 202 while the air trap is inverted. When priming, the air trap 202 fills with working fluid, with the working fluid flowing into the air trap via the input tubing. The output tubing extends into the air trap 202, such that is opening is positioned at the end of the air trap that is opposite the end having the output 256. Therefore, during priming, when the air trap is in an inverted position, as working fluid fills the air trap 202, air at the highest point of the air trap 202 is pushed out of the air trap through the output tubing, which is also located at the highest point of the air trap. The air is then pushed through tubing 208, and back to the fluid source or fluid bag, where it is held in the top portion of the bag. The air trap holder 206 holds the air trap 202 during priming while allowing fluid to flow through the input 254 and output 256 tubing without pinching or crimping the tubes. The air trap holder 206 allows a user to prime the fluid loop without requiring the caregiver to hold the air trap 202 in an inverted orientation. This frees up the caregiver's hands, so that they can attend to the patient, or handle other treatment preparations. The air trap holder makes setup and priming of the system easier and more intuitive. It also reduces the time to complete priming and setup because the user is able to quickly locate the holder and commence priming more rapidly than when using systems that do not have an air trap holder. This allows the caregiver to provide the patient with temperature management treatment as quickly and efficiently as possible.

Once the fluid loop 201 is primed, the air trap 202 is overturned and positioned in an air trap receptacle 230. The air trap receptacle 230 is shaped to accept the air trap and hold the air trap (full of working fluid) in an upright orientation in which the input 254 and output 256 of the air trap are above the chamber of the air trap.

The air trap receptacle 230 of the console 102 interface 104 may include an optical sensor 260. The optical sensor 260 is configured to sense a threshold amount of air present in the air trap 202 cylinder when the air trap cylinder is positioned in an optical pathway of the optical sensor within the air trap receptacle 230. An alarm may be activated when an amount of air in the air trap 202 cylinder exceeds the threshold amount of air. The alarm can be audible, and/or displayed on the user interface (e.g., user interface 106) of the console 102. The alarm can cause one or more actions to occur by a controller 262 of the temperature management system 100. For example, the alarm can cause the controller 262 to stop the temperature management treatment of the patient until the air is reduced below the threshold or removed entirely from the fluid loop 201.

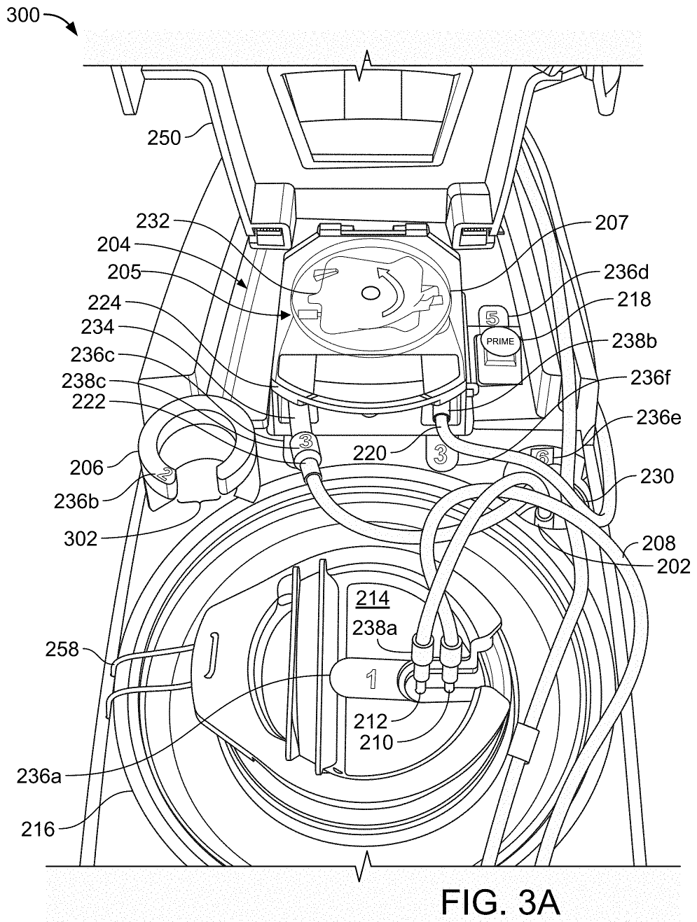

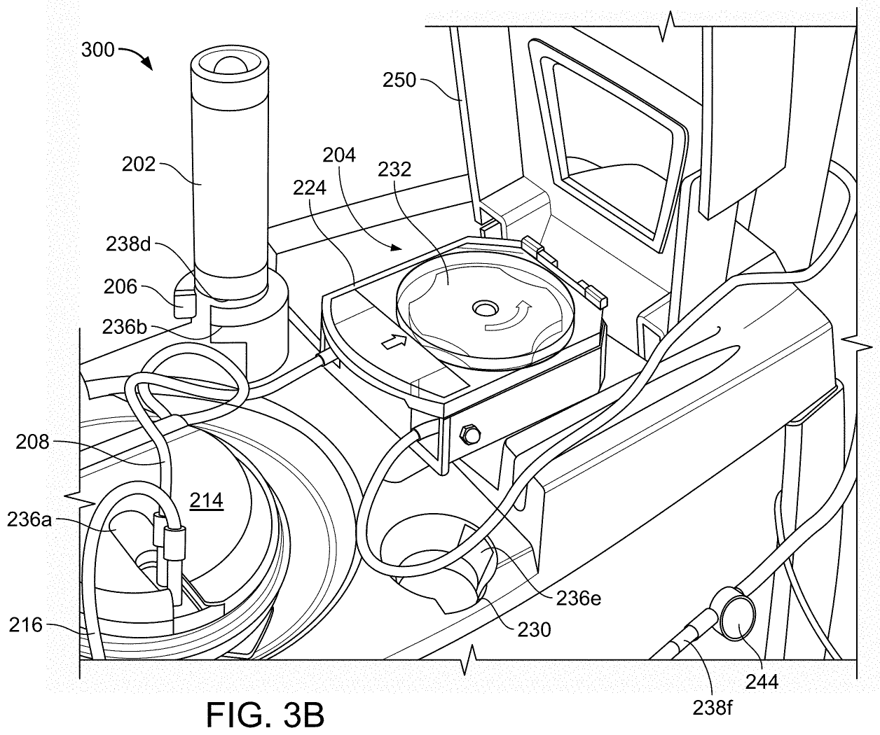

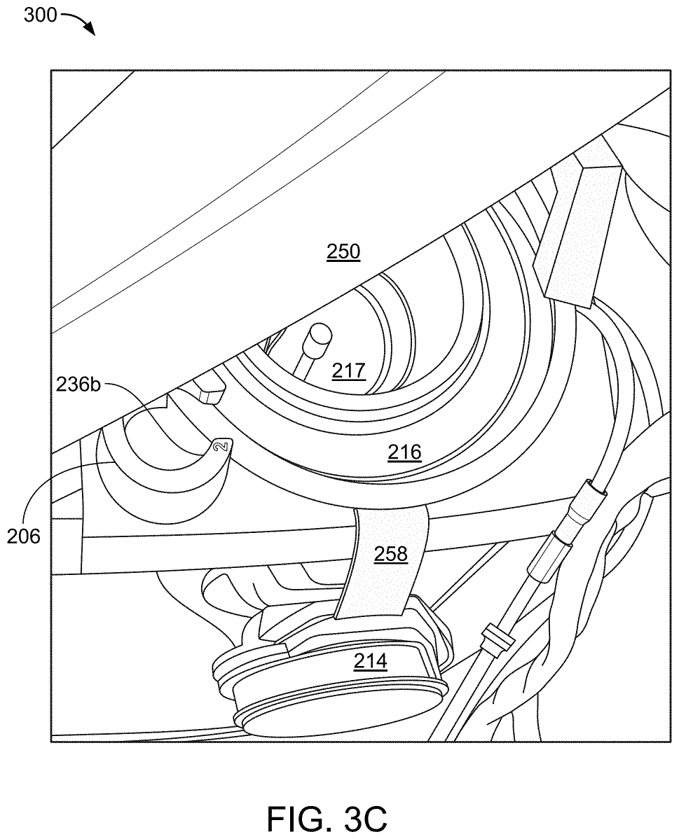

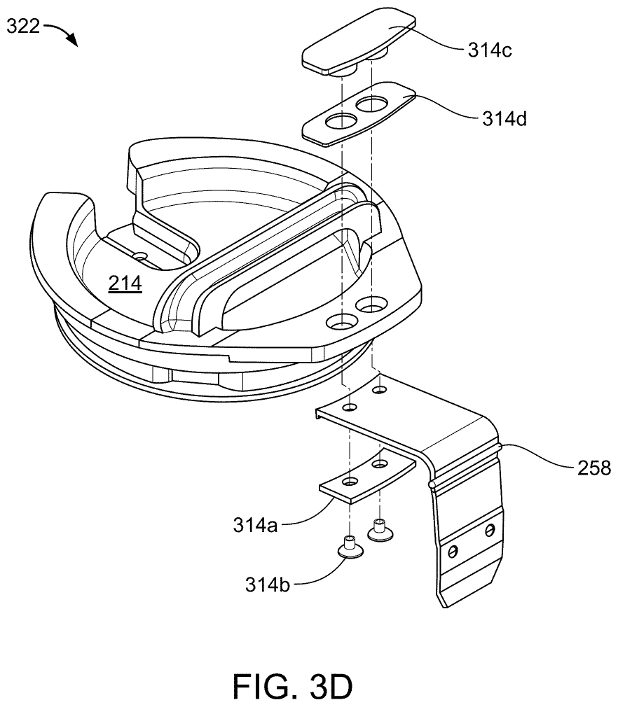

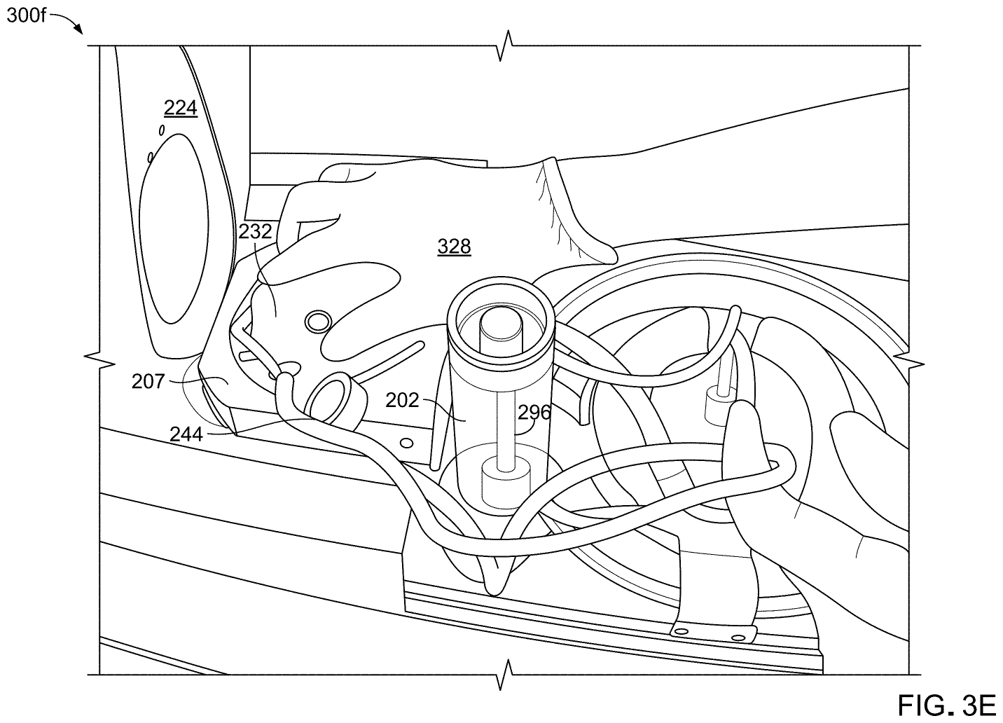

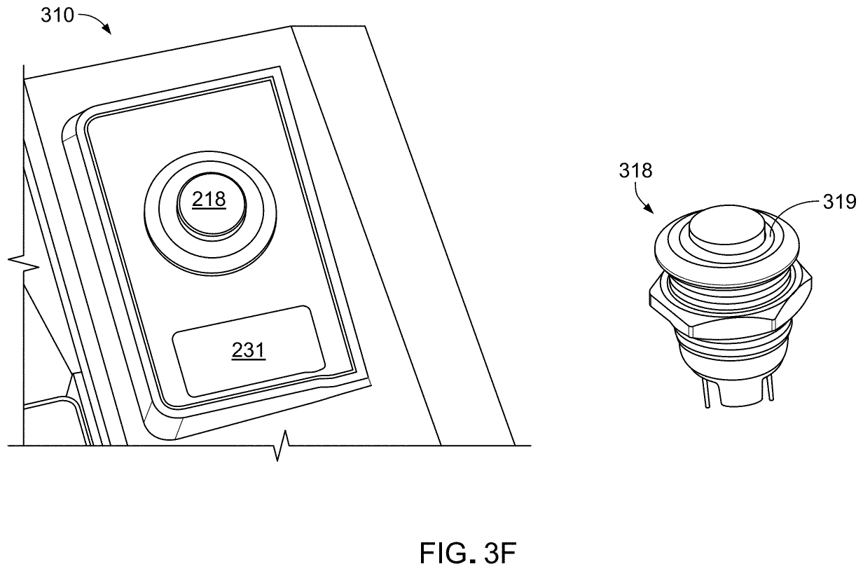

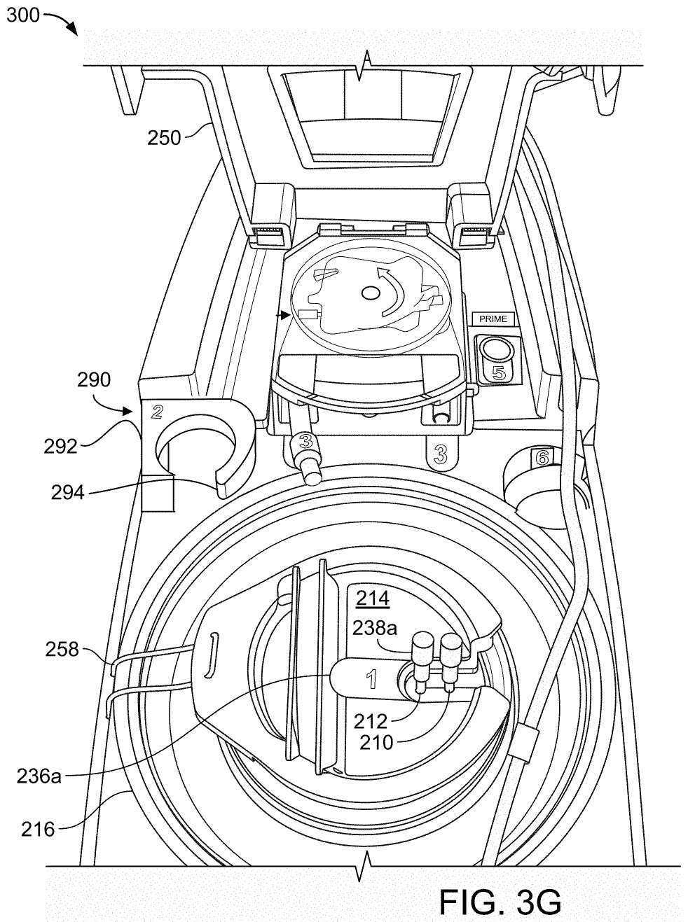

The heat exchange bath 216 is filled with a bath liquid. The liquid can include water, glycol, or other coolant. The heat exchange bath 216 is configured to be warmed or cooled to provide heating or cooling to the working fluid of the fluid loop 201, which is in thermal contact with the heat exchange liquid, and thus the heat exchange device 110 and the patient. The heat exchange bath 216 is configured to receive the coil 242 that is fluidly coupled to, and makes up part of, the heat exchange fluid loop 201. Generally, the heat exchange bath 216 includes an insulated cavity or chamber configured to receive the coil 242. A temperature of the cavity or chamber is controlled by the controller 262 of the temperature management system 100. The cavity or chamber is covered by a bath cap 214. The cap may be attached to the heat exchange bath by a tether 258. The tether 258 prevents the cap 214 from being lost, removed, or from falling into the cavity chamber of the heat exchange bath 216. In some implementations, another retaining device can be used to couple the cap 214 to the heat exchange bath 216, e.g., a wire or strap. The tether 258 or strap can include wires as shown in FIG. 3A. In some implementations, the tether 258 can be a solid strap.

The coil 242 includes a thin tubular structure that is shaped to form a relatively long passageway in a relatively small volume envelope. Generally, the coil 242 forms a helix structure with many loops or windings. This allows a long passageway to be submersed in a bath fluid. The bath fluid warms or cools the coil 242, thereby warming or cooling the working fluid flowing through the coil. The relatively long passageway of the coil helps allow the working fluid to be cooled or warmed to the desired temperature (e.g., approximately the bath temperature) by the time the working fluid has been pumped through the coil 242. Generally, the coil 242 is made of a material (e.g., metal) that is highly thermally conductive.

The working fluid is pumped through heat exchange fluid loop 201 and through the coil 242, which is immersed in bath liquid (e.g., coolant) within the heat exchange bath 216. As the working fluid flows through the coil 242, the working fluid is in thermal communication with the bath liquid (e.g., coolant), and exchanges heat with the bath liquid, resulting in a cooling or warming of the working fluid to a desired temperature. The temperature of the coolant in the heat exchange bath is controlled by the console, e.g., by exchanging heat with a refrigerant flowing through a refrigerant loop within the console. The coil increases a surface area of the fluid loop 201 that is exposed to the coolant in the heat exchange bath 216 such that the working fluid may be quickly cooled or warmed.

As previously stated, the bath cap 214 covers the heat exchange bath 216 to ensure that a desired temperature is maintained in the heat exchange bath. The cap 214 has one or more openings through which an input port 212 and output port 210 of the coil may extend for connecting the coil with tubing 208 of tubing assembly. Generally, tether or retaining device includes a strap that prevents the bath cap from being separated from the heat exchange bath 216. In some implementations, the bath cap 214 is physically coupled to the console 102 when the bath cap is not closing off the heat exchange bath 216 cavity (also called a chamber).

The tubing 208 of the fluid loop 201 is routed to the heat exchange device 110 from the heat exchange bath 216. The tubing 208 can be covered by at least one sleeve 240. The sleeve 240 may include a polyurethane material. The sleeve 240 prevents the tubing 208 from knotting, crimping, etc. during operation of the temperature management system 100. The sleeve can help improve fluid loop and tubing 208 management, e.g., by helping to avoid tangling of the tubing. While the sleeve 240 is shown in FIG. 2 between the heat exchange bath 216 and the heat exchange device 110, the sleeve 240 can be positioned elsewhere in the fluid loop 201 where there is tubing 208. The sleeve may provide some insulation around the tubing 208, and may reduce condensation. Additional sleeves can also be added at other portions of the tubing 208. In some implementations, the tubing 208 is disposable for an intravascular fluid loop.

A priming actuator 218 is provided to prime the fluid loop 201 prior to heating or cooling of the patient. Priming the fluid loop 201 includes removing air from the fluid loop 201 and filling the fluid loop 201 with working fluid. To prime the fluid loop 201, the air trap 202 is positioned in a first inverted orientation on the air trap holder 206, as previously described. The inverted orientation of the air trap causes air bubbles in the fluid loop 201 to be collected in a top portion of the air trap 202, where they are removed through the tubing 208, and pushed into the fluid source or bag 248, where they are trapped in the top portion of the source/bag, thereby removing air from the fluid loop 201.

The priming actuator 218 is actuated and released to cause the pump 204 to pump working fluid through the fluid loop 201 for a set amount of time (e.g., a minute, two minutes, etc.) to ensure that all air has been removed from the fluid loop 201. The priming actuator 218 is configured to activate a priming sequence in response to actuation. For example, the priming actuator 218 can include a button, switch, or other device that can be actuated. The priming sequence terminates after a predetermined amount of time after actuation without the user being required to continue to press or activate the actuator 218. For example, the priming actuator 218 is configured to activate the priming sequence in response to a single press and release of the priming actuator. In some implementations, an indicator is located on the priming actuator for instructing a user when or how to initiate priming, as subsequently described.

Priming of the fluid loop 201 can be confirmed by referencing a flow detector or an air detector 244. The air detector 244 comprises a pinwheel. Once the fluid loop 201 is primed, the optical pinwheel of the air detector 244 rotates in response to liquid flowing through the fluid loop and through the air detector to provide a visual confirmation of a primed fluid loop, and clearance of any air bubbles.

The heat exchange device 110 is connectable to the fluid loop 201 by connectors 203. In some implementations, the heat exchange device 110 is connected to the heat exchange fluid loop 201 after priming is completed. Various connectors may be used, e.g., the connectors 203 can include ZOLL's proprietary non-standard Lure connectors. The heat exchange device 110 can include a catheter (as previously described). In some implementations, the heat exchange device 110 includes a surface pad or other device configured for placement externally on a patient.

In an aspect, the hardware components 200 of the temperature management system 100 are configured to provide for ease and timing of setup for priming and operation of the fluid loop 201. The temperature management system 100 is configured to reduce the time it takes for a user to interact with the hardware components 200 and to setup or install the fluid loop on the control console. The timing and process for priming the fluid loop 201 is streamlined to enable a user to perform other tasks during priming of the fluid loop 201.

The controller 262 is configured to be in communication with the pump 204 and the priming actuator 218 to control priming of the fluid loop 201. As previously described, the pump 204 includes one or more rollers 205 and a raceway 207 and is configured to receive the pump tube 234 of the fluid loop between the one or more rollers and the raceway as previously described. The pump 204 is connected to the air trap 202, which is configured to remove air from the fluid loop 201. The priming actuator 218 is configured to generate a signal in response to a press and release of the priming actuator, e.g., a single press and release. A signal is generated after a predefined debounce period. For example, the debounce period may be about 100-200 milliseconds (ms). A processor (e.g., a controller 262), a memory storing instructions, and associated circuitry communicatively are coupled to the priming actuator 218. The processor is configured to receive the signal from the priming actuator 218, and in response to receiving the signal, perform one or more of the following operations. The operations may include activating the pump 204 to compress the tube 234 between the one or more rollers 205 and the raceway 207 to cause fluid to flow through the fluid loop 201 to remove the air from the fluid loop via the air trap 202. The pump 204 is configured to continue to move fluid through the fluid loop 201 for a predefined period of time after the priming actuator 218 is released. The period of time can be any period of time for removing the air from the fluid loop 201, but generally is about 1 to 3 minutes in duration or around 1.5 minutes. Generally, the priming actuator 218 includes a button. The button can be illuminated upon actuation and remain illuminated until priming of the fluid loop 201 is complete, providing a signal to the user that priming is or is not in progress. The pump 204 is configured to move fluid through the fluid loop 201 until all air in the fluid loop is moved into the air trap 202.

In some implementations, the controller 262 detects a second signal representing a second actuation (e.g., a press and release by a user) of the priming actuator 218 while the pump 204 is moving fluid through the fluid loop 201. The controller 262 causes the pump 204 to stop moving fluid through the fluid loop in response to detecting the second signal.

The one-touch priming feature allows a caregiver to prime the fluid loop without requiring the caregiver to press and hold the priming button as in older systems. This frees up the caregiver's hands, so that they can activate automated priming and leave the system alone, while attending to the patient, or handling other treatment preparations, thereby reducing the time it takes to ready the system for treatment of the patient. The one-touch priming feature makes setup and priming easier and more intuitive. It also helps reduce the time it takes complete priming and setup since the user is able to quickly locate the one-touch actuator and avoid pauses or breaks in the priming sequence due to user fatigue which may arise when using older systems that require the user to manually hold the priming actuator in an actuated or pressed position during the entire priming sequence. The one-touch priming feature allows the caregiver to provide the patient with temperature management treatment as quickly and efficiently as possible.

The air trap holder, discussed previously, in combination with the one-touch priming actuator, allows the caregiver to perform one or more of the following: 1) step back and observe the priming process, making sure that bubbles are exiting the fluid loop properly, 2) ensure that there is no leaking of the fluid loop and/or 3) leave the system unattended, allowing the caregiver to perform other duties for the patient and/or within the hospital.

As previously described, the hardware 200 includes an air detector 244. In certain implementations, the air detector 244 may include a bubble sensor configured to detect the air in the fluid loop 201. In response to detecting air by the bubble sensor of the air detector 244, the controller 262 causes the pump 204 (or the caregiver may cause the controller to cause the pump) to move fluid through the fluid loop 201 until no air is detected by the air detector and/or for a predefined period of time. In some implementations, the controller 262 performs the priming operations using discrete logic devices. In some implementations, the operations are performed by a microprocessor.

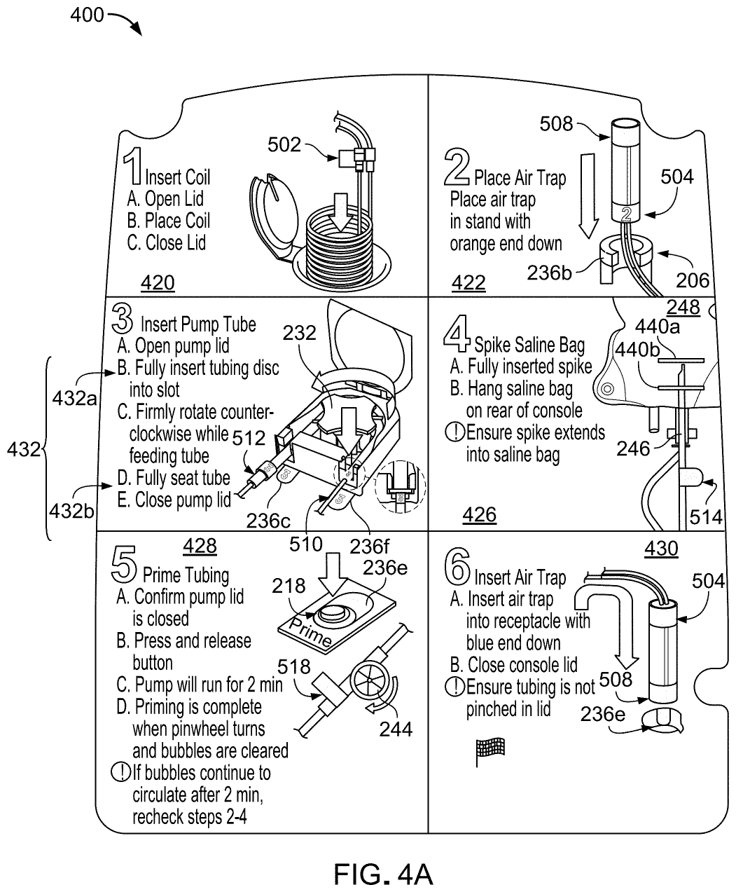

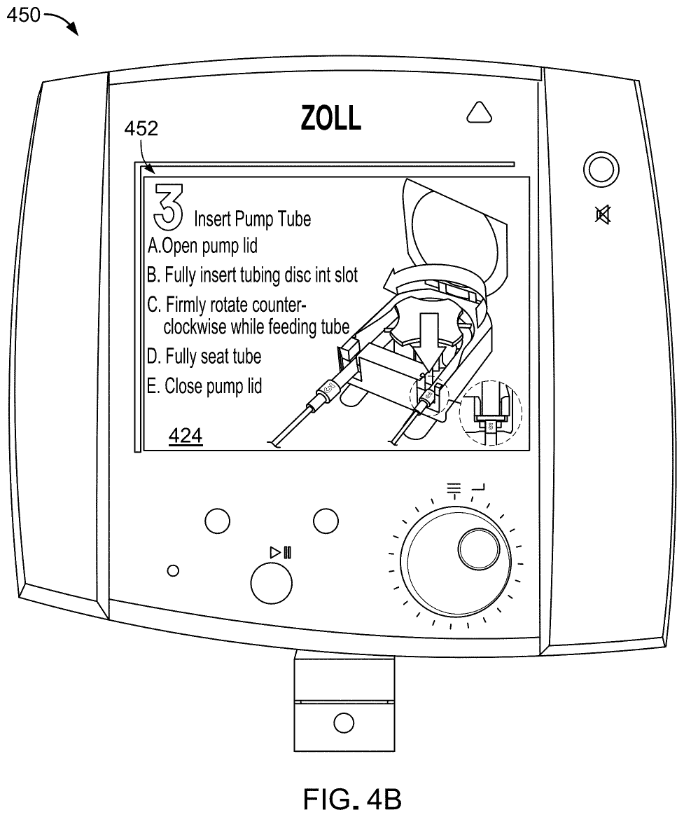

Turning to FIG. 3A, a perspective view is shown of a hardware interface 300 (e.g., hardware interface 104 of FIG. 1 and portions of hardware 200 of FIG. 2). Colored indicators 236a-f, 238a-f (also called markings) are provided on the components of both the fluid loop 201 and the console 102 to assist a user in interfacing, installing, setting up or initializing the heat exchange fluid loop 201 with the console 102. The indicators (e.g., indicators 236a-f) on the console can correspond to instructions provided to the user for setting up the fluid loop 201 (e.g., instructions 400, 450 of FIG. 4A-4B). The console 102 has an openable/closable access cover 250 that enables access to hardware elements of the console that will interface with the fluid loop.

The interface 300 includes indicators for each step of setting up the fluid loop 201 with the console 102 for priming and eventual operation for delivery of temperature management treatment. The first step is inserting the coil 242 into the heat exchange bath 216 in the proper orientation. In this example, an indicator 238a on the tubing 208 connected to the coil 243 corresponds to an indicator 236a on the bath cap 214 of the heat exchange bath 216. The indicators 236a and 238a in this example are each a first color (e.g., blue) with a numerical “1” shown. The coil 242 is oriented such that when inserted into the bath cavity, the tubing 208 connected to an input or output of the coil and having the indicator 238a is positioned adjacent the indicator 236a on the batch cap 214.

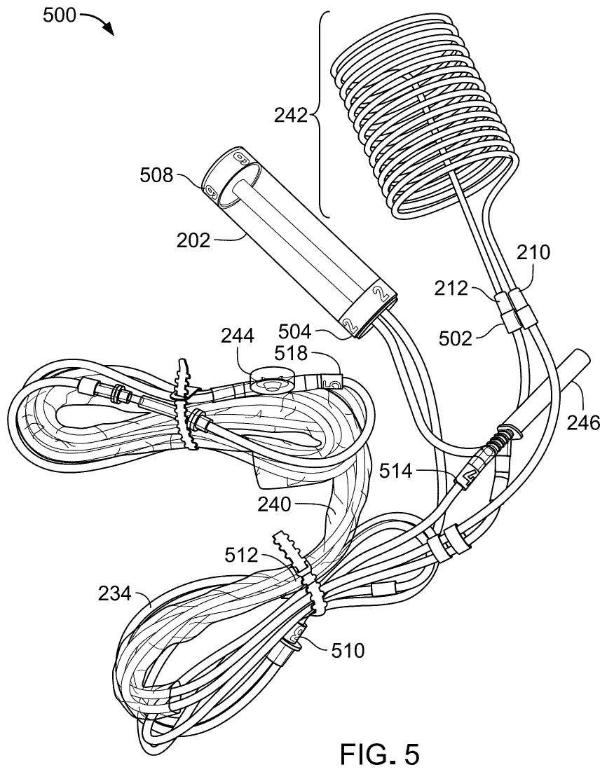

The second step is positioning the air trap 202 in the proper orientation for priming the fluid loop as previously described. The air trap holder 206 is shown with a notch 302 for receiving tubing 208 for the input 254 and output 256 of the air trap 202. A top surface of the air trap holder 206 includes an indicator that is colored a second color (e.g., orange) that is different than the first color (e.g., blue) used on the indicators in the first step. The color difference between the consecutive steps assists a user in quickly identifying the location of the air trap holder 206. A numerical “2” is also placed on the indicator on the air trap holder 206 to indicate to the user that placement of the air trap 202 in the air trap holder is the second step for setting up the fluid loop 201. The air trap 202 (shown in FIG. 5) includes a corresponding indicator 504 that is configured to indicate to the user the inverted orientation of the air trap 202 for placement in the air trap holder 206 at the second step. The air trap 202 is left in the air trap holder 206 until priming is completed. At step six (described below) the air trap 202 is moved to the upright orientation for temperature management treatment and placed in air trap receptacle 230.

The third step includes placing the pump tubing 234 into the pump 204. As subsequently described in further detail, pump tubing 234 is placed between the pump raceway 207 and pump rollers 205. The pump lid 224 is opened by actuating latch 225 to access the raceway 207 and place the tube 234 in the raceway. Indicator 238b on the tubing 208 corresponds to indicator 236f on the console 102. The indicator 238b marks the input 220 of the pump tube 234. The indicators are each the second color (e.g., orange) and have a numerical indicator “3” to show that this is the third step. A corresponding indicator 238c for the other end of the pump tube 234 (the output 222) shows where the output 222 is oriented during step 3. Indicator 238c on the tube 208 near the output 222 of the pump tube 234 corresponds to indicator 236c on the console 102. The indicator pairs for the input 220 (indicators 238b and 236f) and output 222 (indicators 236c and 238c) of the pump tube 234 assist the user to orient the pump tube in the correct direction in the pump 204, in the raceway and around the rollers. The pump tube 234 direction in the pump 204 determines the direction of fluid flow in the fluid loop 201, and therefore the correct orientation is ensured by the indicators 238b-c and 236c-f. In certain implementations, the input and/or output may include a disk or collar that fits into a corresponding slot in the pump, to secure the pump tube in place. While the pump tube 234 is placed in the raceway 207, usually with one of the user's hands, the pump knob 232 is advanced or rotated with the other of the user's hands to rotate the rotor and/or rollers 205 to facilitate positioning of the tube 234 between the raceway and the rollers. For example, the pump knob may be rotated counterclockwise while feeding the tube into the raceway and around the rollers, to load the pump tube onto the pump. In some implementations, the indicators 236c-f can include chevrons showing a direction of the fluid flow. For example, indicator 236f can have chevrons or arrows pointing toward the pump 204 to indicate an input. Indicator 236c can include chevrons or arrows pointing away from the pump to indicate an output.

A fourth step includes spiking the fluid source 248, e.g., a saline or coolant bag, with spike 246. Indicators on the tubing assembly (shown in FIG. 5) assist the user in spiking the fluid source 248. This is described further in relation to FIGS. 4A-4B. The indicator adjacent to the spike is the first color (e.g., blue) and has a numerical indicator “4” to show that this is the fourth step.

A fifth step includes priming the fluid loop 201, as previously described. An indicator 236d on the console shows the location of the priming actuator 218. The indicator is a first color (e.g., blue) and includes a numerical “5” to show that this is the fifth step. Prior to actuating the actuator, the user confirms the pump lid is closed. The actuator is then pressed and released, and the pump will run for a predefined period of time e.g., 1-3 minutes or around 1.5 minutes. Additionally, the air detector 244 is checked, as shown by indicator 238f. Indicator 238f includes a first color (e.g., blue) and a numerical “5” to indicate the fifth step. Priming is complete when the air detector, e.g., pinwheel, turns due to liquid flow through the tubing and all bubbles are cleared. In some implementations, another label “PRIME” may be included on the actuator 218 button.

The priming actuator can be as shown in FIG. 3F. The actuator 318 can be a button that is approximately flush with the surface of the interface 300. The indicator 236d can include an LED indicator on the actuator.

A sixth step includes moving the air trap 202 to the upright orientation, to complete the fluid loop setup and interfacing with the console for use of the fluid loop 201 for temperature management treatment of a patient. The air trap 202 is placed in the air trap receptacle 230. An indicator 236e marks the air trap receptacle 230. The indicator 236e includes a first color (e.g., blue) and a numerical “6” to show that this is the sixth step. The indicator 236e corresponds to an indicator 508 on an end of the air trap 202 (seen in FIG. 5) which is also blue and marked with the numerical “6”. The indicator 236e is generally a different color than indicator 236b on the air trap holder 206 to assist a user in distinguishing between the air trap holder and air trap receptacle.

The holder 206 can have various shapes. The holder 206 can have a circular, ring configuration (as shown in FIG. 3A). In some implementations, one or more corners of the holder are squared. In some implementations, the air trap holder has resilient and/or flexible portions and/or rigid portions, as described in relation to FIG. 3G.

Turning to FIG. 3B, interface 300 is shown with, the air trap 202 inverted and positioned in the air trap holder 206. In this inverted position, indicator 238d on the air trap is near the air trap holder 206 which has corresponding indicator 236b. As previously described, indicators 236b and 238d have a same color (e.g., orange) and number. The orange color highlights the position to assist a user in placing the air trap 202 in the proper orientation during priming at step 2, as shown by the numerical “2” in each of indicators 238d and 236b.

As described, the indicators 236a-f can be numbered (e.g., sequentially) to assist with quickly configuring the fluid loop 201 with the console in the proper orientation for priming of the fluid loop 201 or for operation during heating and cooling treatment. The indicators 236a-f of the console correspond to indicators 502, 504, 508, 510, 512, 514, and 518 of the tubing assembly, seen in full in FIG. 5. This one-to-one correspondence enables a user to quickly match and interface each component of the tubing assembly 500, in the appropriate positions and orientations, with the components at the interface 104 of the console 102. The indicators allow the caregiver to quickly locate and match portions of the tubing assembly and fluid loop with corresponding portions of the console, thereby allowing the caregiver to quickly install the fluid loop and set up the system for priming and operation. The indicators help reduce setup time significantly compared to systems that do not have such indicators. This allows the caregiver to provide the patient with temperature management treatment as quickly and efficiently as possible.

FIG. 3C shows a view of interface 300 in which the bath cap 214 is off of the heat exchange bath 216. A bath cavity 217 is shown into which the coil 242 is configured for placement during priming and operation of the fluid loop 201. The tether 258 ensures that the cap 214 is not lost, dropped, misplaced, or otherwise removed from the interface 300. The tethering of the bath cap to the console allows the user to have both hands free to continue set up and/or operation of the temperature management system, rather than using one hand to hold a removed bath cap.

FIG. 3D shows a schematic view of a bath cap assembly 322. The cap assembly 322 includes the cap 214 and the tether 258. The cap 214 can be circular to mate with the heat exchange bath cavity 217 of the console interface 104. The cap 214 is coupled to the tether using fixing elements including a bracket 314c and screws 314d. The tether is coupled to the console using fixing elements including a bracket 314a and screws 314b. Thus, the cap 214 is movably coupled to the console interface 104 by the tether 258.

FIG. 3E shows a perspective view of interface 300 including the pump 204 and the air trap 202 in the air trap holder 206. A user 328 can adjust or rotate the pump knob 232 once pump lid 224 is opened. As previously described, the user 328 can move the rollers (not shown) along raceway 207 to insert the pump tube 234 into the pump 204, between the rollers and the raceway.

FIG. 3F shows an example priming actuator module 310. The priming actuator 218 is seated near a label 321 reading “PRIME.” The actuator 218 can include a button 318 configured with a debounce mechanism (e.g., a debounce switch or timer). In some implementations, the button includes a light emitting diode (LED) feedback indicator 319. The LED indicator 319 lights during the priming period.

FIG. 3G shows an example of the interface 300 of FIG. 3A including an air trap holder 290. In FIG. 3G, the heat exchange fluid loop is partially shown. While the air trap holder 206 in FIG. 3A includes two gripping elements or fingers, which may be symmetrical and/or generally rigid, the air trap holder 290 includes a single gripping element or finger 294. The finger 294 may by resilient and/or flexible and a more rigid portion 292 may be provided. The holder 290 is configured to accommodate air traps having different diameters, while gripping the air trap to secure it in place. For example, the resilient gripping element or finger 294 can flex away from this rigid portion to allow an air trap (e.g., air trap 202) to be slotted into place in the holder 290. Once the air trap is slotted into the holder 290, the gripping element or finger 294 flexes or returns back toward the rigid portion 292 to secure around the diameter of the air trap and hold the air trap in place. The gripping element or finger 294 thus flexes around the diameter of an air trap and can snugly holds air traps of different diameters in place.

FIG. 5 shows a tubing assembly 500 of the fluid loop for interfacing with the hardware interface 104 of the console. The tubing assembly 500 is generally disposable and configured to be removable from the interface 104 of the console. The tubing assembly 500 includes the air trap 202, the coil 242, the pump tube 234, the sleeve 240, the air/bubble detector 244, the spike 246, and the tubing 208 connecting each of these components.

The tubing assembly 500 includes indicators 502, 504, 508, 510, 512, 514, and 518 that correspond with indicators 236a-f on the console interface 104. Indicator 502 corresponds with indicator 238a of FIG. 3A. Indicator 510 corresponds with indicator 238b of FIG. 3A. Indicator 512 corresponds with indicator 238c of FIG. 3. Indicator 504 corresponds to indicator 238d of FIG. 3B. Indicator 508 corresponds to indicator 238e of FIG. 3B. Each of indicators 502, 504, 508, 510, 512, 514, and 518 may correspond to printed instructions 400 or graphic instructions 450 on the user interface 106, described in relation to FIGS. 4A-4B.

The input 212 of the coil 242 includes indicator 502. Indicator 502 is a first color (e.g., blue) and includes a numerical “1” indicator. Indicator 502 corresponds to indicator 236a on the bath cap 214. Indicator 502 and indicator 236a are a same, first color (e.g., blue) and include a numerical “1”, indicating that this is the first step of coupling the tubing assembly of the fluid loop to the console. The indicator 502 thus specifies the orientation, positioning, and timing for setting up and/or operating the coil 242 during setup, priming and operation of the fluid loop 201 and temperature management system.

The air trap 202 includes indicator 504. Indicator 504 corresponds to indicator 236b of the air trap holder 206. Indicator 504 and indicator 236b are a same, second color (e.g., orange) and include a numerical “2” indicating that this is the second step of coupling the tubing assembly of the fluid loop with the console The second color of indicator 504 contrasts with the first color (e.g., blue) of indicator 508 on an opposing end of the air trap 202. Indicator 508 corresponds to the indicator 236e on the air trap receptacle. Both indicators 508 and 236e are a first color (e.g., blue) and include a numerical “6” indicating that this is the 6th step of coupling the tubing assembly of the fluid loop to the console. The indicators 508 and 504 thus specify the orientation, positioning, and timing for setting up and/or operating the air trap 202 during setup, priming and operation of the fluid loop 201 and temperature management system.

The pump tube 234 includes an input indicator 510 on the input 220 of the pump tube and an output indicator 512 on the output 222 of the pump tube. As previously described, tubing assembly 500 indicator 510 corresponds to indicator 236f of the console interface 104, and tubing assembly indicator 512 corresponds to indicator 236c of the console interface. The indicators 510, 236f are a second color (e.g., orange) and have a numerical “3.” The indicators 512, 236c are a first color (e.g., blue) and include a numerical “3.” This indicates that positioning each of these indicators relative to one another is part of step 3 of coupling the tubing assembly of the fluid loop to the console. The indicators 510, 512 also indicate the proper orientation of the pump tube 234 in the pump. This ensures that the pump 204 moves working fluid in the correct direction through the tubing assembly 500. The indicators 510 and 512 of the pump tube 234 thus specify the orientation, positioning, and timing for setting up and/or operating of the pump tube during setup, priming and operation of the fluid loop 201 and temperature management system.

The spike 246 has an indicator 514 on an output of the spike. The indicator 514 is a first color (e.g., blue) and has a numerical “4.” This indicates that using the spike to puncture the fluid source bag is the fourth step of coupling the tubing assembly of the fluid loop to the console.

The air detector 244 has an indicator 518 near it. The indicator 518 is a first color (e.g., blue) and has a numerical “5.” This indicates that checking the air detector for air bubbles is the fifth step of coupling the tubing assembly of the fluid loop to the console.

As previously stated, each indicator on the tubing assembly 500 of the fluid loop 201 of the set of indicators corresponds to a corresponding indicator on the console interface 104 or elsewhere in the temperature management system in one or more of size, shape, color, or numerical digit. Each indicator of the set of indicators 236a-f is proximate the corresponding indicator on the tubing assembly 500 of the fluid loop 201 when the fluid loop is coupled to the console 102. In some implementations, the second or different or specific color is indicative of a high-priority action for initializing the fluid loop for operation. In some implementations, a size or shape of at least one indicator is different from one or more other indicators of the set of indicators, the size or shape being indicative of a high-priority action for initializing the fluid loop for operation.

The tubing assembly 500 may include a heat exchange coil 242 configured to be disposed in a heat exchange bath 216 as described previously. The tubing assembly 500 may also include a spike 246. The spike 246 may include an elongate body extending from a base portion of the spike. The elongate body includes a first lumen configured to remove fluid, e.g., saline, from a fluid bag into the fluid loop and a second lumen configured to return liquid or air to the fluid bag from the fluid loop. The base portion includes a grip having an angled profile to facilitate ease of insertion of the elongate body into the fluid bag. The spike 246 is described further in relation to FIG. 6A.

As discussed herein, the fluid loop 201 may include an air trap 202 e.g., a cylinder, configured to trap air present in the fluid loop. The fluid loop 201 includes a pump tube 234 configured to interface with a fluid pump to circulate fluid through the fluid loop. The fluid loop 201 includes a plurality of flexible tubes 208 configured to connect the coil, the spike, the air trap, the pump tube, and a heat exchange device. The fluid loop 201 includes indicators 502, 504, 508, 510, 512, 514, and 518 on the fluid loop configured to match to one or more indicators on a console for ease of installing the fluid loop. In some implementations, the coil 242 is metallic. In some implementations, a sleeve 240 is placed around one or more tubes of the plurality of flexible tubes 208. The sleeve is configured to reduce or prevent condensation forming on the one or more tubes caused by fluid in the fluid loop 201. The one or more indicators comprise a first indicator 502 proximate the coil, the first indicator corresponding to an indicator on a bath cap of the heat exchange bath; a second indicator 504 on a first end of the air trap; a third indicator 508 on a second end of the air trap; a fourth indicator 510 proximate a first end of the pump tube; a fifth indicator 512 proximate a second end of the pump tube; and a sixth indicator 514 proximate the spike. In some implementations, the second indicator 504 includes a second color, and wherein the third indicator 508 includes a first color that is different than the second color, the second color corresponding to an air trap holder 206 indicator 236b on the console 102, and the first color corresponding to an air trap receptacle 230 indicator 236e on the console. In some implementations, the fourth indicator 510 comprises a second color, wherein the fifth indicator 512 includes a first color that is different than the second color, the second color corresponding to a pump input indicator 236f on the console 102, and the first color corresponding to a pump output indicator 236c on the console.

FIG. 4A shows a graphical interface with instructions 400 configured for placement on the temperature management system. The instructions 400 show graphic depictions corresponding to the indicators 236a-f of the console interface 104 and the indicators 502, 504, 508, 510, 512, 514, and 518 of the tubing assembly 500 of the fluid loop 201. In some implementations, the instructions 400 may be attached or adhered to the console, e.g., to an underside of the console access cover. The instructions may be paper instructions or a label. Each indicator of the set of indicators 502, 504, 508, 510, 512, 514, and 518 and 236a-f corresponds to the series of instructions including depictions of each of the indicators.

Instruction 420 represents a first step of installing the heat exchange fluid loop coil 242 into the heat exchange bath 216 chamber and closing the cap of the chamber. Indicator 502 is shown in instruction 420.

Instruction 422 represents a second step of placing the air trap 202 into the air trap holder 206 in a first orientation. Indicator 236b is shown in instruction 420 on the holder 206, corresponding indicator 504 is shown on the air trap 202, and indicator 508 is shown for a second orientation of the air trap 202 on another end of the air trap opposing indicator 504.

Instruction 424 represents a third step of installing the pump tube 234 by operation of the pump knob 232. Arrows show how to turn the knob 232. The indicators 510, 512 on the pump tube 234 are shown in instruction 424 next to corresponding indicators 236c, 236f on the console interface 104 to orient the pump tube 234 in the proper direction within the pump. The pump tube may include a collar or disc, which can be received in a corresponding slot in the console interface for securing the pump tube in place.

Instruction 426 represents a fourth step of inserting the spike 246 into a fluid bag to fluidly couple the fluid loop to the fluid source 248 (e.g., a saline bag). The indicator 514 near the spike 246 is shown. Horizontal lines 440a-b show a distance range for inserting the spike 246 into the source 248.

Instruction 428 represents a fifth step of pressing and releasing a priming actuator 218 to activate a priming sequence in response to actuation (after confirming that the pump lid is closed). The priming sequence terminates after a predetermined amount of time. Indicator 236e on the console 104 next to the priming actuator is shown including the numerical “5.” Instruction 428 also represents the step of checking the air detector 244 to confirm that no air remains in the fluid loop 201. Indicator 518 next to the air detector 24 on the tube assembly 500 is also shown, including the numerical “5.”

Instruction 430 represents a sixth step of placing the air trap 202 into the air trap receptacle 230 in the second orientation. Indicator 508 is shown corresponding to indicator 236e of the receptacle 230.

In addition to pictorial representations of the indicators 236a-f of the console interface 104 and corresponding indicator's 502, 504, 508, 510, 512, 514, and 518 of the tubing assembly 500 of the fluid loop 201, instructions 420, 422, 424, 426, 428, and 430 may each include text (e.g., text 432) describing the action(s) to be performed for the respective step. In some implementations, some text 432a-b describing an especially important action can be colored a different color (e.g., orange for text 432a or blue for text 432b) to differentiate the step from other text (e.g., white text) and highlight the step to the user. In certain implementations, the instructions may include pictorial representations, text or a combination thereof.

FIG. 4B shows an example of a graphical user interface 450 comprising a series of instructions for setting up a heat exchange fluid loop. The series of instructions may include one or more of the instructions described previously. In certain implementations, the instructions may instruct the user on performing at least the following setup steps: installation of the at least a portion of the heat exchange fluid loop 201 in the heat exchange cavity 216 and closing of the heat exchange cavity, placement of the air trap into the air trap 202 holder in the first orientation, and installation of the pump tube 234 by operation of the pump knob 232. In some implementations, the graphical user interface 450 comprises a static display of the series of instructions. In some implementations, the graphical user interface 450 comprises a user interface for displaying and interacting with the series of instructions. In some implementations, the graphical user interface 450 comprises a user interface for displaying and interacting with the series of instructions, and the instructions comprise icons or animations. In FIG. 4B, an example instruction 424 from FIG. 4A is shown. The screen 452 of the user interface 450 can show one instruction at a time or more than one instruction at a time. In some implementations, the instruction relevant to the current step for configuring or setting up the fluid loop 201 is shown. The graphical user interface may include a touch screen or other controls.

The graphical interface with instructions provides the caregiver with an easy to access reference with step-by-step guidance for quickly installing the fluid loop and setting up the system for priming and operation. The easy to follow instructions and pictorial representations make setup simple and help reduce setup time significantly compared to systems that do not have such instructions. The instructions are located at the user's eye level, near the console interface or deck during the setup and priming process, e.g., when the instructions are positioned on the underside of the console lid. The instructions are also near eye level when they are displayed via the user interface 106. The instructions are hands-free which helps make the setup process faster and easier, which is important given the stressful environment of treating ED/critical care patients. This allows the caregiver to provide the patient with temperature management treatment as quickly and efficiently as possible.

FIG. 6A shows an example of a spike 600 (e.g., similar to spike 246 previously described). The spike 600 includes an elongate body 604 extending from a base portion 602. The elongate body 604 includes a first lumen 612 configured to remove fluid e.g., liquid such as saline, from a fluid source (e.g., source 248) into the fluid loop 201. The spike 246 includes a second lumen 610 configured to return liquid and/or air to the fluid source (e.g., a saline bag) from the fluid loop 201. In this example, the spike base 602 includes a grip comprising a sequence of ribs 614a-g. In some implementations, the grip can include an angled profile to facilitate ease of insertion of the elongate body into the fluid bag. In that example, each rib 614a-g in the sequence is successively larger to cause the angled profile. However, in FIG. 6A, the ribs are sized equally. Grip is improved by a profile of the ribs 614a-g. Each rib is rectangular with a concave arc on each side of the rib for receiving a patient's thumb or finger, for ease of securing the grip between the fingers.

In some implementations, the spike 600 includes a stepped portion 614. The elongate body 604 extends from the stepped portion 614. The elongate body 604 may have a smaller diameter than the stepped portion 614. The stepped portion 614 may have a smaller diameter than the base portion 602.

The elongate body 604 of the spike 600 may include a beveled edge 606 configured to pierce into the fluid bag. The first lumen 612 extends through the base portion 604 to the beveled edge 606. The second lumen 610 extends through the base portion 604 parallel to the first lumen 612 and may be shorter than the first lumen. The first lumen 612 and the second lumen 610 may each include a rounded profile edge to prevent fluid leaking from the base portion 602 during insertion of the elongate body 604 into the fluid bag. E.g., a saline bag may better seal around the rounded profile edge of the lumens when the spike is inserted through a side wall of the bag, into the bag to prevent leaking.

The beveled edge 606 can include a first beveled edge 608 having a first angle. The second lumen 610 then terminates in a second beveled edge 616 having a second angle that may be different from the first angle.

FIG. 6B shows a cross sectional view of the spike 600 showing spike lumens 210, 212. The lumens 210, 212 are curved to prevent leakage when the spike 600 is used to pierce the fluid source 248. In some implementations, the grip 602 of the spike 600 has a rounded edge to assist a user in grasping the spike.

The improved spike designs described herein, in addition to preventing fluid leaking, provide for ease of grip of the spike and ease of insertion of the spike. The grip makes it easier for the caregiver to hold the spike and the beveled edge makes it easier for the spike to be inserted it into a fluid source, e.g., saline bag, in a single motion. This facilitates fast and easy connection of the fluid loop to the fluid source, which allows the caregiver to quickly set up the system for priming and operation, providing the patient with temperature management treatment as quickly and efficiently as possible.

FIG. 7A shows a perspective view of a pump 700 (e.g., similar to pump 204 previously described). The pump 700 includes a rotor, a set of rollers that extend from the rotor, and an arcuate raceway that extends around the set of rollers. The raceway is configured to receive a pump tube of the heat exchange fluid loop. A pump lid 224 is configured to cover the pump. For example, during operation, the pump cover 224 can prevent any foreign object from interfering with operation of the pump. An arrow 708 can show the appropriate direction for advancing or rotating the pump rotor and/or rollers to facilitate installation of the pump tube 234.

The pump 700 includes a pump knob 232, as previously described. The pump knob 232 includes a circumference edge 706 which may have one or more indentations 710a-d. The pump knob 232 may be receivable in the palm of a user's hand and/or receivable by a user's fingers and the circumference edge 706 allows the user ease of grip and rotation of the pump knob 232. The pump knob 232 orients the rollers to enable loading and unloading of the pump tube. Generally, the pump knob 232 extends from an axis 714 of the rotor and rotates around the axis of the rotor. The axis 714 of the rotor is normal to a surface of the knob bordered by edge 706. The surface extends over at least one roller of the set of rollers.

The pump knob circumference edge 706 with one or more indentations 710a-d assists a grip for torquing or rotating the pump knob 232. In some implementations, the pump knob can extend at least 1-2 in from an axis of the rotor. In some implementations, the pump knob requires less than 2 in-lb. of torque or 1-1.5 in-lb of torque to rotate the knob. In some implementations, the pump knob circumference edge 706 includes multiple indentations 410a-d comprising a plurality of finger slots. In some implementations, the pump knob 232 surface comprises a diameter ranging from 2-4 in. In some implementations, pump knob 232 comprises a flat top surface, which is receivable in the palm of the user's hand.

FIG. 7B shows an example profile of a pump knob 750, which can be similar to pump knob 232. Pump knob 750 includes protrusions 752a-b, receivable by a user's fingers, to assist in gripping and torquing the knob. The protrusions 752a-b can extend a distance L from the center axis of the knob. An example range of the distance L is 1-2 inches. In some implementations, the pump knob requires less than 2 in-lb of torque or 1-1.5 inch-pounds (in-lbs.) of torque to rotate the knob. In some implementations, the pump knob 750 surface comprises a diameter ranging from 2-4 in.

The improved pump knob designs described herein provide for ease of grip and intuitive operation to rotate or advance the pump knob and thus the rotor and rollers. This facilitates fast and easy loading or feeding of the pump tube into the pump raceway, between the rollers and raceway, which allows the caregiver to quickly install the fluid loop and set up the system for priming and operation, providing the patient with temperature management treatment as quickly and efficiently as possible.

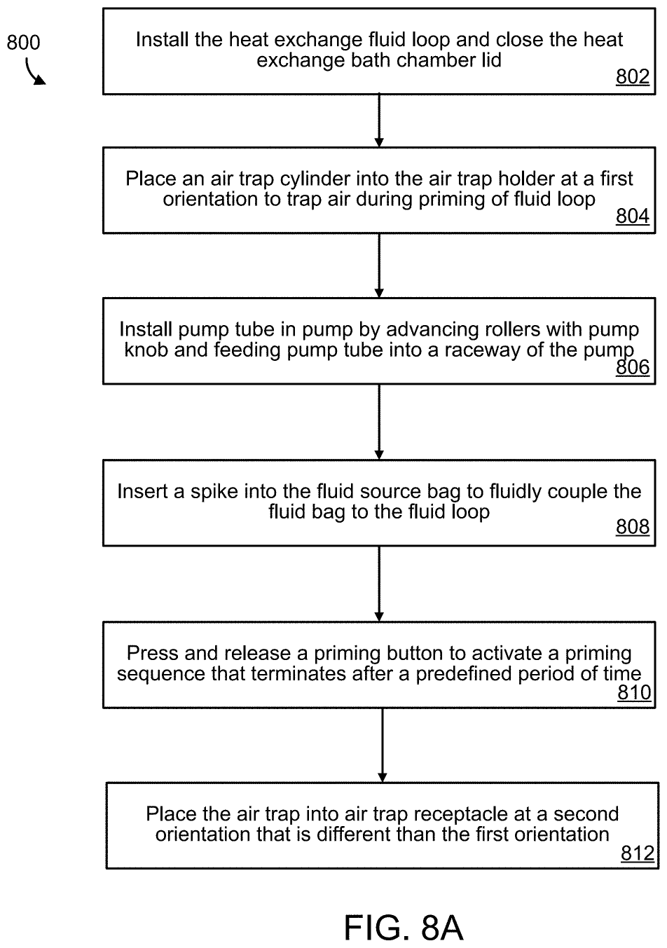

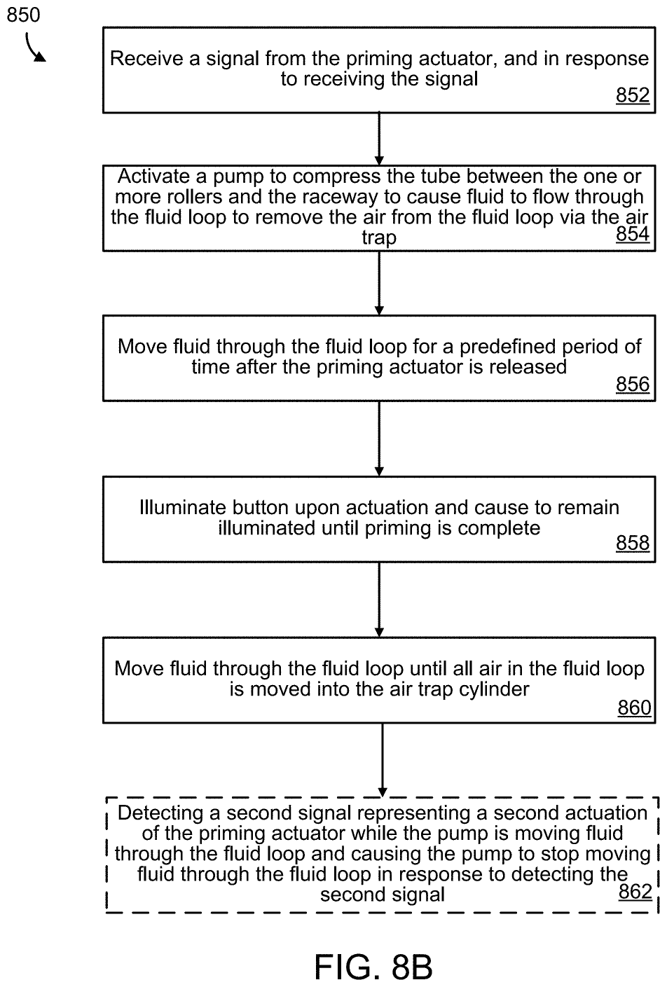

FIG. 8A shows a flow diagram of an example process for treatment of a patient by the temperature management system (e.g., temperature management system 100 of FIG. 1). FIG. 8 shows a process 800 for setting up or installing a fluid loop (e.g., fluid loop 201), the fluid loop may include a tubing assembly, e.g., tubing assembly 500, on a console 102 having a console interface (e.g., interface 104). The process 800 includes a first step (802) including installing the heat exchange fluid loop and closing off the heat exchange chamber. The process 800 includes a second step (804) including placing an air trap cylinder into an air trap cylinder holder in the first orientation. The process 800 includes a third step (806) including installing the pump tube by operation of the pump knob to advance or rotate the rotor and/or rollers. The process 800 includes a fourth step (808) including inserting the spike into a fluid bag e.g., saline bag to fluidly couple the fluid loop to the fluid bag. The process 800 includes a fifth step (810) including pressing and releasing a priming actuator to activate a priming sequence in response to actuation, wherein the priming sequence terminates after a predetermined amount of time. The process 800 includes a sixth step (812) including placing the air trap cylinder into the air trap cylinder receptacle in the second orientation.

FIG. 8B shows a process 850 for priming the fluid loop (e.g., fluid loop 201). Generally, process 850 can be performed by controller 262 of the console 102. The controller 262 is configured to receive (852) a signal from the press and release of a priming actuator. The signal may be generated after a predefined debounce period, e.g., 100-200 milliseconds (ms), and in response to receiving the signal the controller activates (854) the pump to compress the tube between the one or more rollers and the raceway to cause fluid to flow through the fluid loop to remove the air from the fluid loop via the air trap, pushing the air out of the fluid loop and into a fluid source or bag. The pump is configured to continue to move (856) fluid through the fluid loop for a predefined period of time after the priming actuator is released. The priming actuator may be configured to generate a signal in response to a single press and release of the priming actuator.

The predefined period of time can include 1 to 3 minutes or around 1.5 minutes. In some implementations, the priming actuator is a button. The button may be illuminated (858) upon actuation and remain illuminated until priming is complete to indicate that priming is in progress. The pump is configured to move (860) fluid through the fluid loop until all air in the fluid loop is moved into the fluid source or fluid bag e.g., saline bag. In some implementations, the process includes detecting (862) a second signal representing a second actuation of the priming actuator while the pump is moving fluid through the fluid loop causing the pump to stop moving fluid through the fluid loop in response to detecting the second signal.

Some implementations of subject matter and operations described in this specification (e.g., process 800 and/or 850) can be implemented in digital electronic circuitry, or in computer software, firmware, or hardware, including the structures disclosed in this specification and their structural equivalents, or in combinations of one or more of them. For example, in some implementations, the processor of the temperature management system can be implemented using digital electronic circuitry, or in computer software, firmware, or hardware, or in combinations of one or more of them.

Some implementations described in this specification (e.g., the processor of the temperature management system, etc.) can be implemented as one or more groups or modules of digital electronic circuitry, computer software, firmware, or hardware, or in combinations of one or more of them. Although different modules can be used, each module need not be distinct, and multiple modules can be implemented on the same digital electronic circuitry, computer software, firmware, or hardware, or combination thereof.

Some implementations described in this specification can be implemented as one or more computer programs, i.e., one or more modules of computer program instructions, encoded on computer storage medium for execution by, or to control the operation of, data processing apparatus. A computer storage medium can be, or can be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate physical components or media (e.g., multiple CDs, disks, or other storage devices).

The term “data processing apparatus” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed for execution on one computer or on multiple computers that are located at one site or distributed across multiple sites and interconnected by a communication network.

Some of the processes and logic flows described in this specification can be performed by one or more programmable processors executing one or more computer programs to perform actions by operating on input data and generating output. The processes and logic flows can also be performed by, and apparatus can be implemented as, special purpose logic circuitry, e.g., an FPGA (field programmable gate array) or an ASIC (application specific integrated circuit).

Processors suitable for the execution of a computer program include, by way of example, both general and special purpose microprocessors, and processors of any kind of digital computer. Generally, a processor will receive instructions and data from a read only memory or a random access memory or both. A computer includes a processor for performing actions in accordance with instructions and one or more memory devices for storing instructions and data. A computer may also include, or be operatively coupled to receive data from or transfer data to, or both, one or more mass storage devices for storing data, e.g., magnetic, magneto optical disks, or optical disks. However, a computer need not have such devices. Devices suitable for storing computer program instructions and data include all forms of non-volatile memory, media and memory devices, including by way of example semiconductor memory devices (e.g., EPROM, EEPROM, flash memory devices, and others), magnetic disks (e.g., internal hard disks, removable disks, and others), magneto optical disks, and CD-ROM and DVD-ROM disks. The processor and the memory can be supplemented by, or incorporated in, special purpose logic circuitry.

To provide for interaction with a user, operations can be implemented on a computer having a display device (e.g., a monitor, or another type of display device) for displaying information to the user and a keyboard and a pointing device (e.g., a mouse, a trackball, a tablet, a touch sensitive screen, or another type of pointing device) by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

A computer system may include a single computing device, or multiple computers that operate in proximity or generally remote from each other and typically interact through a communication network. Examples of communication networks include a local area network (“LAN”) and a wide area network (“WAN”), an inter-network (e.g., the Internet), a network comprising a satellite link, and peer-to-peer networks (e.g., ad hoc peer-to-peer networks). A relationship of client and server may arise by virtue of computer programs running on the respective computers and having a client-server relationship to each other.

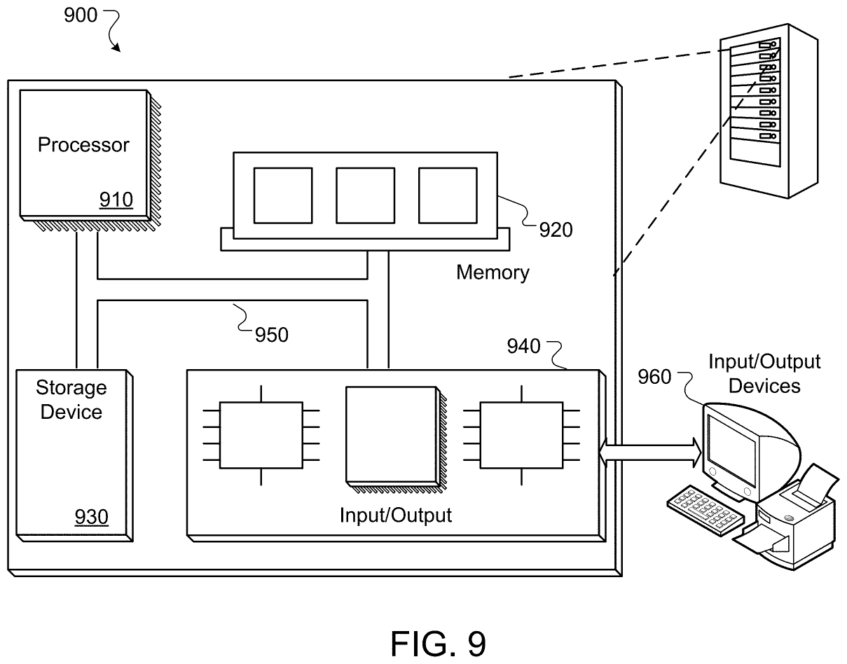

FIG. 9 shows an example computer system 900 that includes a processor 9100, a memory 920, a storage device 930 and an input/output device 940. Each of the components 9100, 920, 930 and 940 can be interconnected, for example, by a system bus 950. The processor 9100 is capable of processing instructions for execution within the system 900. In some implementations, the processor 9100 is a single-threaded processor, a multi-threaded processor, or another type of processor. The processor 9100 is capable of processing instructions stored in the memory 920 or on the storage device 930. The memory 920 and the storage device 930 can store information within the system 900.

The input/output device 940 provides input/output operations for the system 900. In some implementations, the input/output device 940 can include one or more of a network interface device, e.g., an Ethernet card, a serial communication device, e.g., an RS-232 port, and/or a wireless interface device, e.g., an 802.11 card, a 3G wireless modem, a 4G wireless modem, a 5G wireless modem, etc. In some implementations, the input/output device can include driver devices configured to receive input data and send output data to other input/output devices, e.g., keyboard, printer and display devices 960. In some implementations, mobile computing devices, mobile communication devices, and other devices can be used.

While this specification contains many details, these should not be construed as limitations on the scope of what may be claimed, but rather as descriptions of features specific to particular examples. Certain features that are described in this specification in the context of separate implementations can also be combined. Conversely, various features that are described in the context of a single implementation can also be implemented in multiple embodiments separately or in any suitable sub-combination.

A number of embodiments have been described. For example, the detailed description and the accompanying drawings to which it refers are intended to describe some, but not necessarily all, examples or embodiments of the system. The described embodiments are to be considered in all respects only as illustrative and not restrictive. Nevertheless, it will be understood that various modifications may be made without departing from the spirit and scope of the data processing system described herein. Accordingly, other embodiments are within the scope of the following claims.

1. An improved temperature management system for ease and timing of setup by a user comprising:
a heat exchange fluid loop;
a control console comprising:
a heat exchange bath configured to receive at least a portion of the heat exchange fluid loop in a heat exchange cavity and couple with a bath cap to close off the heat exchange cavity;
a pump comprising:
a rotor;
a set of rollers that extend from the rotor;
an arcuate raceway that extends around the set of rollers, wherein the raceway is configured to receive a pump tube of the heat exchange fluid loop;
a pump knob configured to be receivable in a user's hand and allow for ease of grip and rotation of the pump knob by the user, the pump knob configured to orient the rollers to enable loading and unloading of the tube;
an air trap coupled to the fluid loop and configured to couple with the control console via an air trap holder in a first orientation during priming of the heat exchange fluid loop and couple with the control console via an air trap receptacle in a second orientation during operation of the temperature management system; and
a graphical interface comprising a series of instructions for setting up the heat exchange fluid loop, wherein the series of instructions instruct the user on performing at least the following setup steps: installation of the at least a portion of the heat exchange fluid loop in the heat exchange cavity of the heat exchange bath and closing of the heat exchange cavity, placement of the air trap into the air trap holder in the first orientation, and installation of the pump tube by operation of the pump knob.

2. The system of claim 1, the control console further comprising a set of indicators each indicative of an order for interacting with the fluid loop to set up the fluid loop for operation, the set of indicators comprising:
a first indicator on the bath cap;
a second indicator on the air trap holder;
a third indicator on a pump input;
a fourth indicator on a pump output;
a fifth indicator on the priming actuator; and
a sixth indicator on the air trap receptacle.

3. The system of claim 2, wherein each indicator of the set of indicators corresponds to the series of instructions including depictions of each of the indicators.

4.-8. (canceled)

9. The system of claim 1, wherein the control console further comprises a priming actuator and the series of instructions instruct the user on performing the following additional setup step:
pressing and releasing a priming actuator to activate a priming sequence in response to actuation, wherein the priming sequence terminates after a predetermined amount of time.

10. The system of claim 1, wherein the fluid loop is coupled to a spike, and the series of instructions instruct the user on performing the following additional setup step:
inserting the spike into a fluid bag to fluidly couple the fluid loop to the fluid bag.

11. The system of claim 1, wherein the series of instructions instruct the user on performing the following additional setup step:
placing the air trap into the air trap receptacle in the second orientation.

12. The system of claim 1, wherein the series of instructions instruct the user on performing the setup steps in the following order:
a first step of installing the heat exchange fluid loop and closing off the heat exchange bath;
a second step of placing the air trap into the air trap holder in the first orientation;
a third step of installing the pump tube by operation of the pump knob;
a fourth step of inserting the spike into a fluid bag to fluidly couple the fluid loop to the fluid bag;
a fifth step of pressing and releasing a priming actuator to activate a priming sequence in response to actuation, wherein the priming sequence terminates after a predetermined amount of time; and
a sixth step of placing the air trap into the air trap receptacle in the second orientation.

13. The system of claim 1, wherein the air trap holder is keyed to orient the air trap in the first orientation.

14. The system of claim 1, further comprising a pump lid, the pump lid configured to prevent manipulation of the pump rotor during operation of the pump, the pump lid comprising a marking indicating a handle to open the pump lid from the pump.

15. The system of claim 1, the pump further comprising a pump latch, wherein a first portion of the latch is coupled to a pump body and a corresponding second portion of the latch is coupled to the pump lid, wherein the first portion and the second portion connect to hold the lid closed, and wherein less than 5 lbs of force is required to separate the first portion from the second portion to open the lid.

16. The system of claim 1, wherein the bath cap is coupled to the heat exchange bath by a retaining device.

17. The system of claim 14, wherein the retaining device is a strap that prevents the bath cap from being separated from the heat exchange bath.

18. The system of claim 1, wherein the bath cap is physically coupled to the control console when the bath cap is not closing off the heat exchange cavity.

19. The system of claim 1, further comprising a priming actuator configured to prime the fluid loop by causing the pump to circulate fluid through the fluid loop.

20. (canceled)

21. The system of claim 19, wherein the priming actuator is configured to activate a priming sequence in response to actuation, and wherein the priming sequence terminates after a predetermined amount of time.

22. The system of claim 21, wherein the priming actuator is configured to activate the priming sequence in response to a single press and release of the priming actuator.

23.-31. (canceled)

32. The system of claim 1, wherein the pump knob extends from an axis of the rotor and rotates around the axis of the rotor, the axis of the rotor being normal to the flat surface, and wherein the flat surface extends over at least one roller of the set of rollers.

33. The system of claim 1, wherein the pump knob circumference edge with multiple indentations is configured to assist a grip for torquing the pump knob.

34. (canceled)

35. The system of claim 1, wherein the pump knob requires less than 2 in-lbs. of torque to rotate the knob.

36. (canceled)

37. (canceled)

38. The system of claim 1, wherein the pump knob comprises a flat top surface, which is receivable in the palm of the user's hand.

39.-69. (canceled)
